(19) United States
(12) Patent Application Publication
(10) Pub. No.: US 2026/0138970 A1
(43) Pub. Date: May 21, 2026

(54) MODULATORS OF THE CALCIUM SIGNALING CASCADE

(71) Applicants: FONDAZIONE TELETHON ETS, Roma (RM) (IT); FONDAZIONE ISTITUTO ITALIANO DI TECNOLOGIA, Genova (GE) (IT)

(72) Inventors: Luis Juan Vicente GALIETTA, Genova (GE) (IT); Michele GENOVESE, Roma (RM) (IT); Tiziano BANDIERA, Genova (GE) (IT); Fabio BERTOZZI, Genova (GE) (IT); Francesco BERTI, Genova (GE) (IT)

(73) Assignees: FONDAZIONE TELETHON ETS, Roma (RM) (IT); FONDAZIONE ISTITUTO ITALIANO DI TECNOLOGIA, Genova (GE) (IT)

(21) Appl. No.: 19/121,969

(22) PCT Filed: Oct. 30, 2023
(86) PCT No.: PCT/EP2023/080271

§ 371 Date: Apr. 17, 2025

(30) Foreign Application Priority Data
Oct. 28, 2022 (IT) 102022000022332

(51) Int. Cl. C07D 403/04; A61K 31/351; A61K 31/416; A61K 31/4545; A61K 31/506; A61K 45/06; C07D 239/42; C07D 309/06; C07D 405/12; C07D 405/14

(52) U.S. Cl. CPC C07D 403/04; A61K 31/351; A61K 31/416; A61K 31/4545; A61K 31/506; A61K 45/06; C07D 239/42; C07D 309/06; C07D 405/12; C07D 405/14

(57) ABSTRACT

The present invention is directed to novel modulators of the Ca²⁺-signaling cascade, acting as potentiators of the P2RY2 purinergic receptor and of the inositol triphosphate receptors, ITPRs. The compounds are useful in the treatment of diseases and conditions in which modulation of Ca²⁺-signaling plays a role, such as in some forms of spinocerebellar ataxia, and in diseases and conditions benefiting from mucosal hydration, as for example respiratory diseases and conditions, dry eye, xerostomia.

### FIELD OF THE INVENTION

The present invention refers to pharmacological modulators of the Ca²⁺ signaling cascade as a novel therapeutic strategy for a series of human diseases. The compounds of the invention modulates the Ca²⁺ signaling pathway by acting at different levels. In particular, they are allosteric potentiators of the purinergic receptor P2RY2 and the inositol triphosphate receptor type 1 (ITPR1), two targets for which there were no available modulators acting with this mechanism. The compounds are useful for the treatment of diseases and conditions in which potentiation of Ca²⁺ signaling has a beneficial effect, such as in some forms of spinocerebellar ataxia, and in conditions benefiting from mucosal hydration, such as dry eye and chronic obstructive respiratory diseases.

### BACKGROUND OF THE INVENTION

The Ca²⁺ signaling cascade is a complex array of molecular mechanisms that transduce extracellular chemical and mechanical stimuli into mobilization of Ca²⁺ from intracellular stores or Ca²⁺ influx from the extracellular space. Typically, chemical stimuli (hormones, neurotransmitters, exogenous substances) bind to plasma membrane G protein-coupled receptors (GPCRs) that activate phospholipase C (PLC) enzymes (1). Activated PLCs catalyze the breakdown of phosphatidylinositol bisphosphate (PIP₂) into inositol 1,4,5-trisphosphate (IP₃) and diacylglycerol (DAG). IP₃ binds to receptors (inositol triphosphate receptors, ITPRs) localized in the membrane of the endoplasmic reticulum and working as Ca²⁺-permeable channels (2). There are three types of ITPRs (ITPR1-3) that can form homo-tetramers or hetero-tetramers with different intrinsic characteristics and tissue/subcellular distribution (3). Activation of ITPRs upon IP₃ binding triggers Ca²⁺ release from the endoplasmic reticulum with local elevation of Ca²⁺ (4) that can in turn modulate the function of a large series of effectors including other ion channels, Ca²⁺-regulated enzymes, cytoskeleton proteins, and transcription factors (1). An additional mechanism of intracellular Ca²⁺ elevation is influx through plasma membrane proteins belonging to different families: i) voltage-dependent Ca²⁺ channels; ii) chemical-, temperature-, and stretch-sensitive TRP channels; iii) store-operated ORAI1-3 channels (5). TMEM16A (official name: ANO1) is a Ca²⁺-activated Cl⁻ channel expressed in different types of tissues and cell types, including airway surface epithelia, exocrine glands, smooth muscle, nociceptive neurons, and olfactory receptors (6-9). TMEM16A is typically activated by purinergic agents (UTP, ATP), histamine, acetylcholine, and other Ca²⁺ agonists through the GPCR-PLC-ITPR cascade. Potentiation of TMEM16A-dependent Cl⁻ transport in airway epithelia could potentially overcome the defect in Cl⁻ secretion that occurs in cystic fibrosis (CF), thus restoring mucociliary clearance (10). TMEM16A-dependent anion transport can be detected in a very sensitive and rapid way with a cell-based assay employing the halide-sensitive yellow fluorescent protein (HS-YFP) (11,12). For this reason, within the present invention, it was decided to use cells co-expressing TMEM16A and HS-YFP in a high-throughput assay to find direct TMEM16A potentiators but also small molecule modulators of Ca²⁺ signaling. Actually, pharmacological modulators of the Ca²⁺ signaling pathway, acting on specific components of the cascade, are important as both research tools and possible therapeutic agents to treat a variety of human diseases. By screening a maximally-diverse chemical library, the inventors of the present invention found compounds acting with specific mechanisms, including potentiation of purinergic receptor P2Y2 (P2RY2) and ITPR1 proteins.

### SUMMARY OF THE INVENTION

The present invention relates to the identification and development of novel compounds which are positive modulators of Ca²⁺ signaling and which are therefore for use in the treatment of diseases and conditions in which potentiation of Ca²⁺ signaling has a beneficial effect, such as in some forms of spinocerebellar ataxia, and in diseases and conditions benefiting from mucosal hydration, as for example respiratory diseases and conditions, dry eye, xerostomia.

It is therefore an object of the invention a positive modulator of Ca²⁺ signaling having the general formulae (I), (II), (III) or (IV) as indicated below:

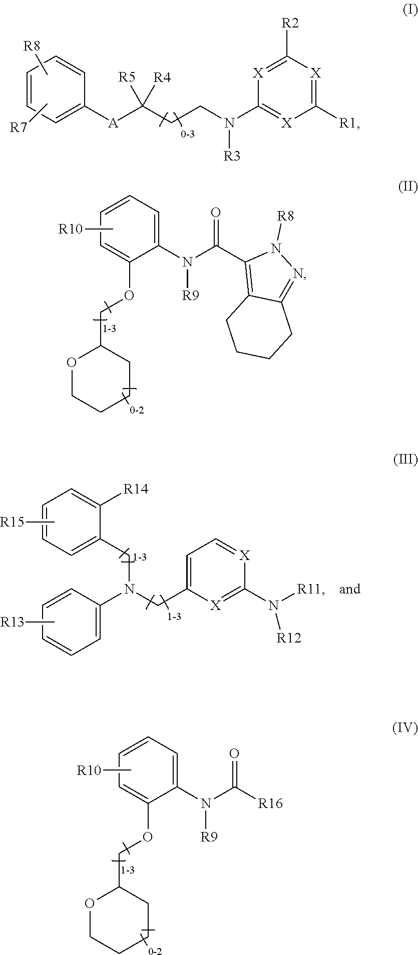

wherein in general formula (I):
X is C or N, with the proviso that at least one X is N; A is O, S, NH, N—C₁₋₆alkyl, preferably A is O;
R₁ is an heteroaromatic ring optionally substituted with one or more substituents selected from C₁₋₆alkyl, C₃₋₆cycloalkyl, halogen, OH, OC₁₋₆alkyl, C₁₋₆fluoroalkyl, OC₁₋₆fluoroalkyl, CN; preferably R₁ is an heteroaromatic ring optionally substituted with one or more substituents selected from C₁₋₆alkyl, C₃₋₆cycloalkyl, halogen, OH, OC₁₋₆alkyl;
R₂ is H, C₁₋₆alkyl, C₃₋₆cycloalkyl, NH₂, NHC(═O)C₁₋₆alkyl, NHC₁₋₆alkyl, N(C₁₋₆alkyl)₂;
R₃ is H, C₁₋₆alkyl, C₃₋₆cycloalkyl;
R₄ and R₅ are each independently H or C₁₋₆alkyl; or R₅ is linked to R₃ to form a 5- or 6-membered heterocyclic ring;
R₆ and R₇ are each independently H, C₁₋₆alkyl, OC₁₋₆alkyl, C₃₋₆cycloalkyl, halogen, CN, C₁₋₆fluoroalkyl, OC₁₋₆fluoroalkyl; preferably R₆ and R₇ are each independently H, C₁₋₆alkyl, OC₁₋₆alkyl, C₃₋₆cycloalkyl, halogen, CN;
   wherein in general formula (II):
R₈ and R₉ are each independently selected from H, C₁₋₆alkyl and C₃₋₆cycloalkyl;
R₁₀ is H, C₁₋₆alkyl, OC₁₋₆alkyl, C₃₋₆cycloalkyl, halogen, CN;
   wherein in general formula (III):
X is C or N, with the proviso that at least one X is N;
R₁₁ and R₁₂ are C₁₋₆alkyl and C₃₋₆cycloalkyl or R₁₁ and R₁₂ form together with the N to which are linked a C₄₋₆heterocycloalkyl ring; preferably R₁₁ and R₁₂ form together with the N to which are linked a pyrrolidine ring;
R₁₃ and R₁₅ are each independently selected from H, C₁₋₆alkyl, OC₁₋₆alkyl, C₃₋₆cycloalkyl, halogen, CN, C₁₋₆fluoroalkyl, OC₁₋₆fluoroalkyl; preferably R₁₃ and R₁₅ are each independently selected from H, C₁₋₆alkyl, OC₁₋₆alkyl, C₃₋₆cycloalkyl, halogen, CN;
R₁₄ is H, C₁₋₆alkyl, (CH₂)ₙOH, (CH₂)ₙNH₂, (CH₂)ₙOC₁₋₆alkyl, (CH₂)ₙNHC₁₋₆alkyl, (CH₂)ₙN(C₁₋₆alkyl)₂, wherein n is 1 to 6;
   wherein in general formula (IV):
R₉ is H, C₁₋₆alkyl or C₃₋₆cycloalkyl;
R₁₀ is H, C₁₋₆alkyl, OC₁₋₆alkyl, C₃₋₆cycloalkyl, C₁₋₆ fluoroalkyl, OC₁₋₆ fluoroalkyl, halogen, CN or an heteroaromatic ring;
R₁₆ is a mono- or bicyclic cycloalkyl, mono- or bicyclic heterocycloalkyl, mono- or bicyclic cycloalkenyl or mono- or bicyclic heterocycloalkenyl optionally substituted with one or more substituents each independently selected from C₁₋₆alkyl, OC₁₋₆alkyl, C₃₋₆cycloalkyl, halogen, aromatic or heteroaromatic ring;
   and any tautomeric form, enantiomer, isotopic variant, salt and solvate thereof.

In a preferred embodiment the present invention is directed to a compound of general formula (IV) wherein R₁₆ is selected from cyclopropyl, cyclobutyl, cyclopentyl, cyclohexyl, cycloheptyl, cyclooctyl, tetrahydrofurane, tetrahydropyrane, piperidine, morpholine, spiro[2.3]hexane, bicyclo[3.1.0]hexane, 5-oxaspiro[2.4]heptane, 2-azabicyclo[2.2.1]heptane, bicyclo[2.2.1]heptene each of said ring being optionally substituted with one or more substituents each independently selected from C₁₋₆alkyl, OC₁₋₆alkyl, C₃₋₆cycloalkyl, halogen, OH, CN, optionally substituted phenyl, optionally substituted thiophene, optionally substituted pyridine, optionally substituted pyrrole, optionally substituted pyrazole, optionally substituted imidazole or optionally substituted thiazole.

In a further preferred embodiment the present invention is directed to a compound of general formula (IV) wherein R₁₆ is selected from cyclopropyl, cyclobutyl, cyclopentyl, cyclohexyl, cycloheptyl, cyclooctyl, tetrahydrofurane, tetrahydropyrane, piperidine, morpholine, spiro[2.3]hexane, bicyclo[3.1.0]hexane, 5-oxaspiro[2.4]heptane, 2-azabicyclo[2.2.1]heptane, bicyclo[2.2.1]heptene each of said ring being optionally substituted with one or more substituents each independently selected from C₁₋₆alkyl, OC₁₋₆alkyl, C₃₋₆cycloalkyl, halogen, OH, CN, phenyl, thiophene, pyridine, pyrrole, pyrazole, imidazole or thiazole.

In a preferred embodiment the present invention is directed to a compound of general formula (I) and/or of general formula (II) and/or of general formula (III) and/or of general formula (IV), wherein said compound is characterized in that it is a positive modulator of Ca²⁺ signaling.

In a preferred embodiment, the positive modulator of Ca²⁺ signaling of the invention is selected from the following compounds:

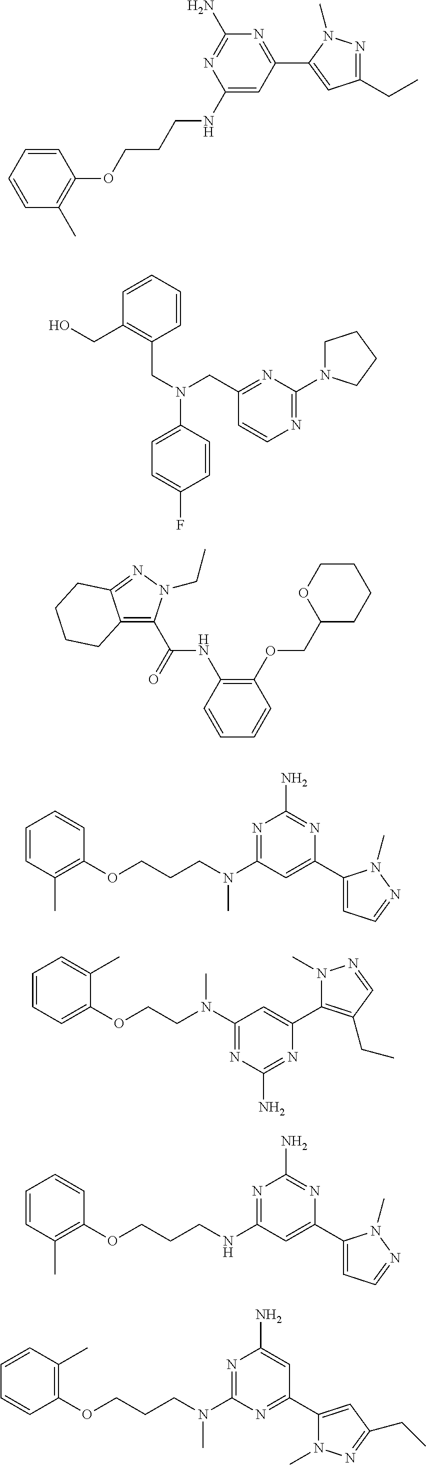

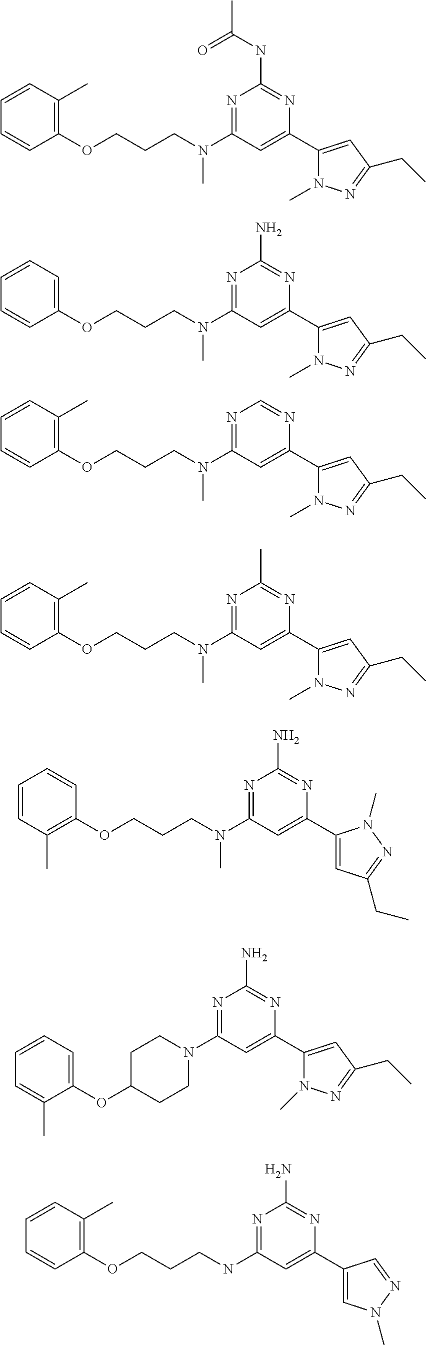

and any tautomeric form, enantiomer, isotopic variant, salt and solvate thereof.

Still preferably the compound is selected from

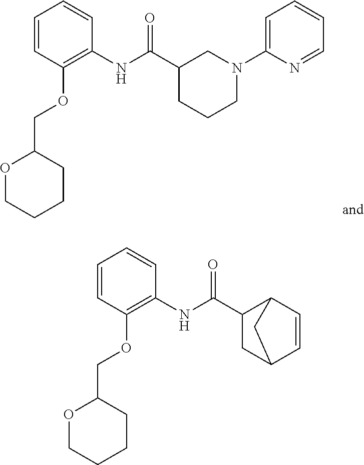 [SMILES: O=C(Nc1ccccc1OCC1CCCCO1)C1CCCN(c2ccccn2)C1 ; O=C(Nc1ccccc1OCC1CCCCO1)C1CC2C=CC1C2]

and any tautomeric form, enantiomer, isotopic variant, salt and solvate thereof.

It is a further object of the invention the positive modulator of Ca²⁺ signaling as defined above being an allosteric potentiator of the purinergic receptor P2RY2 and/or of ITPR, preferably said ITPR is ITPR1.

Preferably, the positive modulator of Ca²⁺ signaling of the invention, or the compound of general formula (I) or of general formula (II) or of general formula (III) or of general formula (IV) as above defined, is for medical use, preferably for use in the treatment and prevention of diseases and conditions affected by modulation of TMEM16A or of other components of the Ca²⁺ signaling cascade, preferably for use the treatment and prevention of diseases and conditions associated with impaired mucociliary clearance, preferably said diseases and conditions being selected from respiratory diseases and conditions, ocular diseases and conditions, spinocerebellar ataxia, dry mouth (xerostomia), intestinal hypermobility, cholestasis, Gillespie syndrome (GLSP; OMIM #206700).

Preferably, the respiratory diseases and conditions are selected from cystic fibrosis, chronic obstructive pulmonary disease (COPD), chronic bronchitis, emphysema, bronchiectasis, including non-cystic fibrosis bronchiectasis, asthma and primary ciliary dyskinesia, respiratory tract infections, lung carcinoma; the spinocerebellar ataxia is spinocerebellar ataxia caused by ITPR1 defective function, preferably spinocerebellar ataxia type 29 (SCA29) or spinocerebellar ataxia type 15 (SCA15);
the dry mouth (xerostomia) results from Sjorgen syndrome, radiotherapy treatment or xerogenic drugs; the intestinal hypermobility is associated with gastric dyspepsia, gastroparesis, chronic constipation or irritable bowel syndrome;
the ocular disease is dry eye disease.

It is a further object of the invention a pharmaceutical composition comprising one or more compounds as defined above together with a pharmaceutically acceptable excipient; preferably said pharmaceutical composition according further comprises one or more additional pharmaceutical agents, preferably selected from mucolytic agents, bronchodilators, antibiotics, anti-infective agents, CTFR modulators and anti-inflammatory agents; still preferably said pharmaceutical composition is for medical use, preferably for use in the treatment and prevention of diseases and conditions affected by modulation of TMEM16A, preferably for use the treatment and prevention of diseases and conditions associated with impaired mucociliary clearance, preferably said diseases and conditions being selected from respiratory diseases and conditions, ocular diseases and conditions, spinocerebellar ataxia, dry mouth (xerostomia), intestinal hypermobility, cholestasis, Gillespie syndrome (GLSP; OMIM #206700); preferably the respiratory diseases and conditions are selected from cystic fibrosis, chronic obstructive pulmonary disease (COPD), chronic bronchitis, emphysema, bronchiectasis, including non-cystic fibrosis bronchiectasis, asthma and primary ciliary dyskinesia, respiratory tract infections, lung carcinoma; preferably the spinocerebellar ataxia is spinocerebellar ataxia type 29 (SCA29) or spinocerebellar ataxia type 15 (SCA15); preferably the dry mouth (xerostomia) results from Sjorgen syndrome, radiotherapy treatment or xerogenic drugs; preferably the intestinal hypermobility is associated with gastric dyspepsia, gastroparesis, chronic constipation or irritable bowel syndrome; preferably the ocular disease is dry eye disease.

The agents of the invention act to potentiate components of the Ca²⁺ signaling cascade including the TMEM16A chloride channel and are useful in the treatment of conditions, which respond to the potentiation of Ca²⁺ signaling, particularly conditions benefiting from mucosal hydration.

As previously indicated, transmembrane member 16A (TMEM16A, also known as Anoctamin-1 (AN01)) is a calcium activated chloride channel expressed in the airway epithelium. Diseases benefiting from potentiation of TMEM16A include diseases associated with the regulation of fluid volumes across epithelial membranes. For example, the volume of airway surface liquid is a key regulator of mucociliary clearance and the maintenance of lung health. The potentiation of TMEM16A by acting on the upstream Ca²⁺ signaling will promote a durable chloride flux from pulmonary epithelia leading to fluid accumulation and mucus hydration on the mucosal side of the airway epithelium thereby promoting mucus clearance and preventing the accumulation of mucus and sputum in respiratory tissues (including lung airways). Such diseases include respiratory diseases, such as chronic bronchitis, chronic obstructive pulmonary disease (COPD), bronchiectasis, asthma, cystic fibrosis, primary ciliary dyskinesia, respiratory tract infections (acute and chronic; viral and bacterial) and lung carcinoma. Diseases sensitive to potentiation of TMEM16A-dependent chloride and fluid secretion also include diseases other than respiratory diseases that are associated with abnormal fluid regulation across an epithelium, perhaps involving abnormal physiology of the protective surface liquids on their surface, e.g., xerostomia (dry mouth) or keratoconjunctivitis sire (dry eye).

Bronchiectasis is the dilation and damage of the large airways of the lungs (bronchi) with loss of the smooth muscle and loss of elasticity of segments of the bronchi. The resultant airway distortion prevents secretions from being adequately cleared from the lung, allowing bacteria to grow and cause recurrent lung infections. The disease may be localized to one area of a lung, or generalized throughout both lungs. Bronchiectasis represents the final common pathway of a number of infectious, genetic, autoimmune, developmental and allergic disorders and is highly heterogeneous in its etiology, impact and prognosis. Increased secretion of anions via potentiation of TMEM16A in lung epithelia will lead to improved hydration of pathologic mucus, resolving the dysregulation of mucociliary clearance by enhancing the clearance and therefore preventing progressive chronic remodeling driven by recurrent exacerbations, chronic infection and mucus dysregulation.

Chronic obstructive pulmonary disease (COPD) is a chronic inflammatory disease of the lung characterized by persistent respiratory symptoms (dyspnea, cough, sputum production) and poorly reversible airflow limitation that is due to airway and/or alveolar abnormalities. Chronic airflow limitation is caused by a mixture of small airways disease (obstructive bronchiolitis) and parenchymal destruction (emphysema). COPD is associated with episodic periods of symptom deterioration termed exacerbations. Exacerbations are important events in the natural history of COPD that drive lung function decline. COPD exacerbations are associated with systemic and pulmonary inflammation and increased levels of inflammatory mediators and cells have been measured in airway tissues e.g. TNF-α, IL-8, IL-6, leukotriene B4, neutrophils, lymphocytes and eosinophils. COPD encompasses a spectrum of diseases, with chronic bronchitis at one end and emphysema at the other, with most individuals having some characteristics of both. Chronic bronchitis, due to mucous hypersecretion and mucociliary dysfunction characterized by chronic cough and sputum, is a key phenotype in COPD subjects with numerous clinical consequences, including an increased exacerbation rate, accelerated decline in lung function, worse health-related quality of life, and possibly increased mortality. COPD patients have decreased mucociliary clearance and increased mucus solids consistent with airway dehydration. Potentiation of TMEM16A will improve airway hydration and potentially act as a surrogate for CFTR-mediated chloride secretion and therefore alter mucus viscosity and enhance mucociliary clearance in COPD.

Asthma is a chronic disease in which inflammation causes the bronchial tubes to narrow and swell, creating breathing difficulties that may range from mild to life-threatening. Asthma includes both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, e.g., anti-inflammatory (e.g., cortico-steroid) or broncho-dilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

In certain embodiments, the present invention provides a method of treating a condition, disease, or disorder associated with the regulation of fluid volumes across epithelial membranes, the method comprising administering a composition comprising a compound of formula (I), of formula (II) or of formula (III) or of formula (IV) to a subject, preferably a mammal, in need of treatment thereof. According to the invention an “effective dose” or an “effective amount” of the compound or pharmaceutical composition is that amount effective for treating or lessening the severity of one or more of the diseases, disorders or conditions as recited above. The compounds and compositions, according to the methods of the present invention, may be administered using any amount and any route of administration effective for treating or lessening the severity of one or more of the diseases, disorders or conditions recited above.

As indicated herein, the compounds are “allosteric potentiators of P2RY2 and ITPR1”, wherein this expression indicates compounds positively modulating intracellular Ca²⁺ concentration via indirect mechanism of action.

As used herein, the expression “positive modulator of Ca²⁺ signaling” refers to a compound which is characterized by being a positive modulator of Ca²⁺ signaling. Preferably said compound modulates the Ca²⁺ signaling pathway by acting at different levels. In particular, it is an allosteric potentiators of the purinergic receptor P2RY2 and the inositol triphosphate receptor type 1 (ITPR1).

As indicated herein, the invention also provides pharmaceutical compositions comprising one or more compounds of this invention and a pharmaceutically acceptable carrier. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions of the invention may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated, or they may be coated by known techniques to mask the unpleasant taste of the drug or delay disintegration and absorption in the gastrointestinal tract and thereby provide a sustained action over a longer period. For example, a water-soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butanediol. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Compounds of the invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound(s) of the invention are employed. For purposes of this application, topical application shall include mouth washes and gargles.

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

The compounds of the invention may be presented in a liposome or other micro particulate or other nanoparticles designed to target the compound. Acceptable liposomes can be neutral, negatively, or positively charged, the charge being a function of the charge of the liposome components and pH of the liposome solution. Liposomes can be normally prepared using a mixture of phospholipids and cholesterol. Suitable phospholipids include phosphatidylcholine, phosphatidylethanolamine, phosphatidic acid, phosphatidylglycerol, phosphatidylinositol. Polyethylene glycol can be added to improve the blood circulation time of liposomes. Acceptable nanoparticles include albumin nanoparticles and gold nanoparticles.

When a compound according to this invention is administered into a human subject, the daily dosage will normally be determined by the prescribing physician with the dosage generally varying according to the age, weight, sex and response of the individual patient, as well as the severity of the patient's symptoms. Generally, dosage levels on the order of from about 0.01 mg/kg to about 150 mg/kg of body weight are useful in the treatment of the above indicated conditions.

As used herein, the term “composition” is intended to encompass a product comprising the specified ingredients in the specified amounts, as well as any product which results, directly or indirectly, from combination of the specified ingredients in the specified amounts.

Another object of the present invention relates to an in vitro method of modulating TMEM16A with the compound of the present invention. This may be useful, for instance, to evaluate whether any given compound potentiates the calcium signaling cascade and therefore modulates the corresponding targets. A further object of the present invention concerns a kit comprising at least one pharmaceutically acceptable vial or container of other type, containing one or more doses of a compound of the invention, including any pharmaceutically acceptable salt, solvate or stereoisomer thereof, or of a pharmaceutical composition of the invention and optionally a) instructions for use thereof in mammals and/or b) an infusion bag or container containing a pharmaceutically acceptable diluent.

In certain embodiments, the compound or the composition of the invention is administered parenterally, intramuscularly, intravenously, subcutaneously, orally, pulmonary, intrathecally, topically, intranasally, or systemically.

In certain embodiments, the patient who is administered the compound or the composition of the invention is a mammal, preferably a primate, more preferably a human.

The compounds of this invention may be administered to mammals, preferably humans, either alone or in combination with pharmaceutically acceptable carriers, excipients or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal and topical routes of administration.

As used herein, the term “prevention” means no disorder or disease development if none had occurred, or no further disorder or disease development if there had already been development of the disorder or disease. Also considered is the ability of one to prevent some or all of the symptoms associated with the disorder or disease. As used herein, any reference to “treatment”/“treating” includes the amelioration of at least one symptom of the disease/disorder to be treated. Such amelioration is to be evaluated in comparison to the same symptom prior to administration of the compound or composition of the invention.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

Compounds of the invention are characterized by molecular structures falling within general formulae (I), (II), (III) or (IV). In the definitions of the substituents, the term “aryl” or “aromatic ring” means a monocyclic or polycyclic aromatic ring comprising carbon atoms and hydrogen atoms. If indicated, such aromatic ring may include one or more heteroatoms, then also referred to as “heteroaryl” or “heteroaromatic ring”, preferably, 1 to 3 heteroatoms, independently selected from nitrogen, oxygen, and sulfur, preferably nitrogen. As is well known to those skilled in the art, heteroaryl rings have less aromatic character than their all-carbon counter parts. Thus, for the purposes of the present invention, a heteroaryl group need only have some degree of aromatic character. Preferably, the ring component of aryl or heteroaryl groups comprises 5 or 6 members (i.e., atoms). Still preferably, aryl or heteroaryl groups are polycyclic aromatic rings. Illustrative examples of aryl groups are optionally substituted phenyls. Illustrative examples of heteroaryl groups according to the invention include optionally substituted thiophene, oxazole, thiazole, thiadiazole, imidazole, pyrazole, pyrimidine, pyrazine, pyridine and pyridine N-oxide. Thus, examples of monocyclic aryl optionally containing one or more heteroatoms, for example one or two heteroatoms, are a 5- or 6-membered aryl or heteroaryl group such as, but not limited to, phenyl, pyridyl, pyrimidinyl, pyrazinyl, pyridazinyl, pyrrolyl, thienyl, thiazolyl, thiadiazolyl, pyrazolyl, imidazolyl, triazolyl, tetrazolyl, furyl, isoxazolyl, oxadiazolyl and oxazolyl. Examples of polycyclic aromatic ring, optionally containing one or more heteroatoms, for example one or two heteroatoms, are a 8-10 membered aryl or heteroaryl group such as, but not limited to, benzimidazolyl, benzofurandionyl, benzofuranyl, benzofurazanyl, benzopyrazolyl, benzotriazolyl, benzothienyl, benzoxazolyl, benzoxazolonyl, benzothiazolyl, benzothiadiazolyl, benzoxadiazolyl, benzoisoxazolyl, benzoisothiazolyl, indolyl, indolinyl, indolizinyl, indazolyl, isobenzofuranyl, isoindolyl, isoindolinyl, isoquinolyl, quinazolinyl, quinolyl, quinoxalinyl, quinolizinyl, naphthyl, naphthyridinyl and phthalazinyl. Other examples of polycyclic heteroaromatic rings according to the invention are 2H-pyrazolo[3,4-b]pyridine, indazole, 2H-pyrazolo[3,4-c]pyridine, 6H-pyrrolo[3,4-b]pyridine, 6H-pyrrolo[3,4-b]pyrazine, 6H-pyrrolo[3,4-d]pyrimidine, 2H-pyrazolo[3,4-d]pyrimidine, 1,5-naphthyridine, imidazo[1,2-a]pyridine. A preferred aryl according to the present invention is phenyl. A preferred heteroaryl according to the present invention is pyridyl.

The expressions “optionally substituted aryl”, “optionally substituted heteroaryl”, “optionally substituted aryloxy”, “optionally substituted heteroaryl-C₁₋₆alkyl”, “optionally substituted heteroaryl-C₁₋₆alkoxy” generically refer to aryl, heteroaryl or aryloxy groups wherein the aromatic or heteroaromatic ring may be substituted with one or more substituents. Examples of said substituents include alkyl, alkoxy, amino, trifluoromethyl, aryl, heteroaryl, hydroxyl, carboxyalkyl, halogen, cyano and the like, preferably include C₁₋₆alkyl, O—C₁₋₆alkyl, halogen, OH, and cyano.

The expression “heterocycloalkyl” refers to a saturated ring containing at least one heteroatom selected from S, N or O, preferably N. Examples of heterocycloalkyls are aziridine, azetidine, pyrrolidine, piperidine, piperazine, morpholine, azacycloheptane, azacyclooctane and the like. The expression “saturated 5- or 6-membered heterocyclic ring” or the expression “heterocycloalkyl” refer to a saturated 5- or 6-membered ring containing at least one heteroatom selected from S, N or O, preferably N. Examples of said saturated 5- or 6-membered heterocyclic ring are pyrrolidine, piperidine, morpholine, piperazine, tetrahydrofurane, tetrahydropyrane and the like. The expression “heterocycloalkenyl” refers to any of the above heterocyclic ring, bearing a double bond within the ring.

As used herein, “alkyl” is intended to include both branched and straight-chain saturated aliphatic hydrocarbon groups having the specified number of carbon atoms. For example, “C₁₋₆alkyl” is defined to include groups having 1, 2, 3, 4, 5 or 6 carbons in a linear or branched arrangement and specifically includes methyl, ethyl, n-propyl, i-propyl, n-butyl, t-butyl, i-butyl, pentyl, hexyl, and so on. Preferably, “C₁₋₆alkyl” refers to “C₁₋₄alkyl” or “C₁₋₃alkyl”. “C₁₋₄alkyl” is defined to include groups having 1, 2, 3 or 4 carbons in a linear or branched arrangement. As used herein, “cycloalkyl” refers to a saturated carbon ring and “cycloalkenyl” refers to said carbon ring also bearing a double bond within the ring. Said cycloalkyl and cycloalkenyl can be monocyclic or bicyclic systems.

As used herein, “C₁₋₆ fluoroalkyl” refers to a branched and straight-chain saturated aliphatic hydrocarbon groups substituted with one or more fluoride atom. Preferably, “C₁₋₆fluoroalkyl” refers to “C₁₋₂fluoroalkyl”, “C₁₋₃fluoroalkyl” or trifluoromethyl.

As used herein the term tautomer refers to constitutional isomers of organic compounds that readily convert by tautomerization or tautomerism. The interconversion commonly results in the formal migration of a hydrogen atom or proton, accompanied by a switch of a single bond and adjacent double bond. Tautomerism is a special case of structural isomerism, and because of the rapid interconversion, tautomers are generally considered to be the same chemical compound. In solutions in which tautomerization is possible, a chemical equilibrium of the tautomers will be reached. The exact ratio of the tautomers depends on several factors including, but not limited to, temperature, solvent and pH. Exemplary common tautomeric pairs include, but are not limited to, ketone and enol, enamine and imine, ketene and ynol, nitroso and oxime, amide and imidic acid, lactam and lactim (an amide and imidic tautomerism in heterocyclic rings), and open-chain and cyclic forms of an acetal or hemiacetal (e.g., in reducing sugars).

The present invention will be described by means of non-limiting data examples, referring to the following figures:

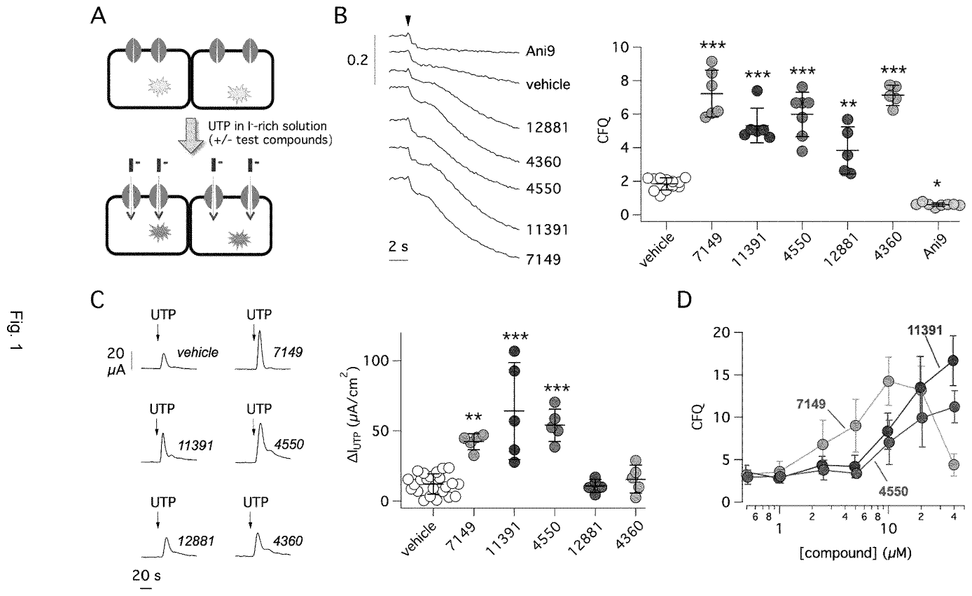

FIG. 1. Identification of Ca²⁺ signaling cascade potentiators by high-throughput screening. (A) Scheme of the screening assay. Fischer rat thyroid (FRT) cells with co-expression of the TMEM16A Cl⁻ channel and HS-YFP (12) were preincubated for 20 min with compounds in 96-well microplates. For the assay, the microplate reader continuously recorded cell fluorescence 2 s before and 12 s after addition of a saline solution containing I⁻ instead of Cl⁻ plus a submaximal UTP concentration (0.25 μM). TMEM16A channel activation by UTP resulted in I⁻ influx and HS-YFP quenching. Presence of an active compound in the well was detected by faster and larger quenching. (B) Detection of active compounds by HS-YFP assay. Representative traces (left) and summary of data (right) obtained for indicated compounds. The scatter dot plot reports activity as Cumulative Fluorescence Quenching (CFQ). CFQ in the time interval between 3 and 13.8 s (representing integration of TMEM16A-dependent I⁻ influx) was quantified according to the formula

$${\sum }_{i = 3}^{13.8} Δ ( Fi - F 0 ) .$$

*, p<0.05; **, p<0.01; ***, p<0.001 vs. vehicle (ANOVA with Dunnett's post-hoc test). (C) Representative traces (left) and summary of data (right) from short-circuit current (Isc) recordings on FRT cells with stable expression of TMEM16A. Cells were briefly pre-incubated with indicated compounds (10 μM) or vehicle and then stimulated with 0.25 μM UTP (on the apical side) to induce TMEM16A-dependent Cl⁻ transport. The scatter dot plot reports the value of maximal UTP effect. The current activated by UTP is significantly enhanced by ARN7149, ARN11391, and ARN4550 compared to vehicle. **, p<0.01; ***, p<0.001 (ANOVA with Dunnett's post-hoc test). (D) Dose-response of ARN7149, ARN11391 and ARN4550 by HS-YFP assay in FRT cells expressing TMEM16A.

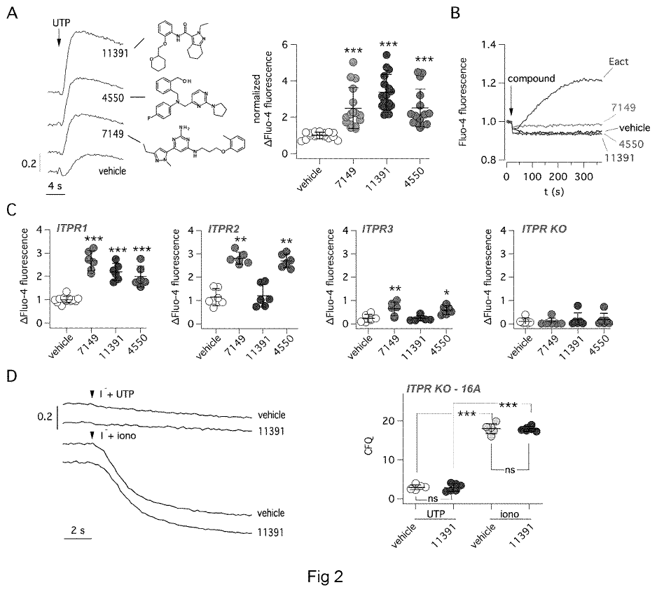

FIG. 2. Effect of active compounds on Ca²⁺ mobilization. (A) Left: representative traces showing effect of 0.25 μM UTP (with/without indicated compounds, 10 μM) on Fluo-4 fluorescence in null FRT cells. The chemical structures of compounds is shown. Right: summary of UTP effect on Fluo-4 fluorescence. The symbols report the normalized maximal change in fluorescence caused by UTP with vehicle or compounds (10 μM). ***, p<0.001 vs. vehicle (ANOVA with Dunnett's post-hoc test). (B) Representative traces showing the time-course of Fluo-4 fluorescence following acute addition (arrow) of vehicle, indicated compounds from the screening (10 μM), or Eact (5 μM) as a TRPV4 agonist. (C) Summary of data obtained with the Fluo-4 assay in HEK293 cells with selective expression of ITPR1, ITPR2, ITPR3 or totally devoid of ITPR expression (ITPR KO). Ca²⁺ elevation was induced by UTP. *, p<0.05; **, p<0.01; ***, p<0.001 vs. vehicle (ANOVA with Dunnett's post-hoc test). (D) Representative traces (left) and summary of data (right) from HS-YFP assay carried out in ITPR-defective HEK293 cells transiently transfected with TMEM16A. Cells were stimulated with 5 μM UTP or 1 μM ionomycin (iono) with/without 10 μM ARN 1391. ***, p<0.001 (ANOVA with Tukey's post-hoc test).

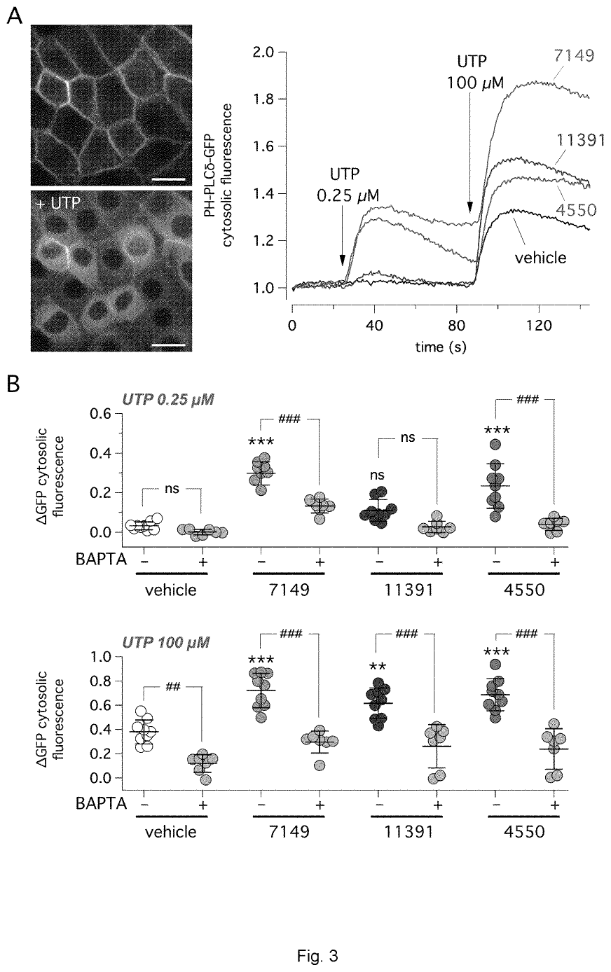

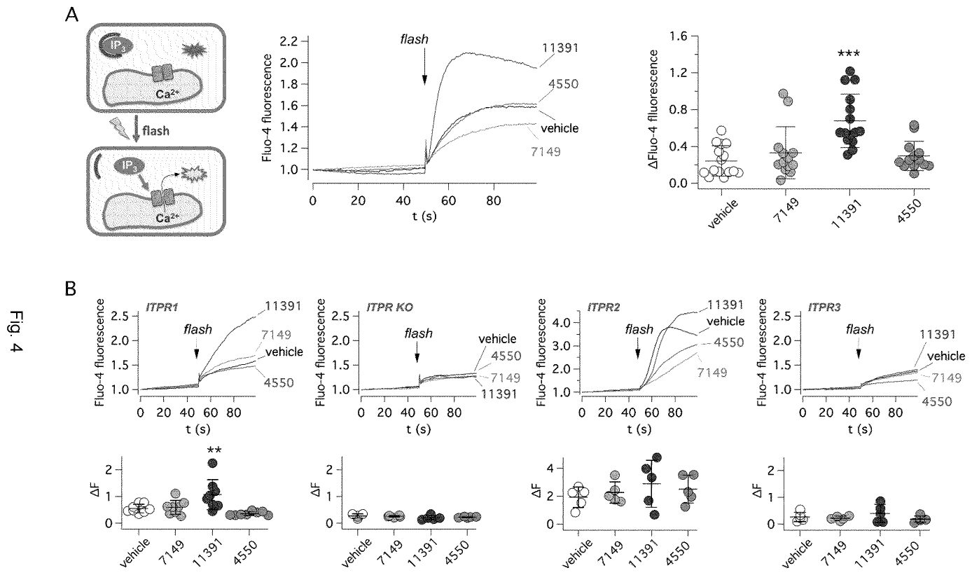

FIG. 3. Effect of active compounds on PLC activity. (A) Representative images (left) and GFP fluorescence traces (right) showing relative changes in cytosolic GFP fluorescence in FRT cells stably expressing PH-PLC6-GFP probe. During the assay, cells were sequentially stimulated with low (0.25 μM) and high (100 μM) UTP concentration. PLC activation, causing PIP₂ breakdown, results in detachment of the probe from the plasma membrane and redistribution to the cytosol. Vehicle or indicated compounds (10 μM) were added to the cells 20 min before the assay. (B) Summary of data showing the increase in cytosolic PH-PLC6-GFP localization elicited by the addition of UTP (0.25 μM, top; 100 μM, bottom) in presence of vehicle or compounds. Where indicated, cells were pre-incubated with the membrane-permeable BAPTA/AM (1,2-Bis(2-aminophenoxy)ethane-N,N,N′,N′-tetraacetic acid tetrakis(acetoxymethyl ester)) (Tocris, catalog number: 2787) to chelate cytosolic Ca²⁺. ***, p<0.001 vs. vehicle without BAPTA. ##, p<0.01; ###, p<0.001 vs. indicated condition. ns: not significant (ANOVA with Tukey's post-hoc test).

FIG. 4. Evaluation of active compounds on ITPR function. (A) Left: scheme of IP₃ uncaging experiments. Cells were loaded with ci-IP₃/PM (6-O-[(4,5-Dimethoxy-2-nitrophenyl)methyl]2,3-O-(1-methylethylidene)-D-myo-Inositol 1,4,5-tris[bis[(1-oxopropoxy)methyl]phosphate]) and Fluo-4. Ca²⁺ release from IP3 sensitive stores was elicited with a light flash. Middle: representative traces from experiments on FRT cells showing intracellular Ca²⁺ increase by ci-IP3 photolysis. Experiments were done in the presence of vehicle or indicated compounds (10 μM). Right: summary of data. Each symbol shows the maximal amplitude of Fluo-4 increase for indicated conditions. ***, p<0.001 vs. vehicle (ANOVA with Dunnett's post-hoc test). (B) IP₃ uncaging experiments in HEK293 cells with expression of a specific ITPR or totally devoid of ITPR expression (ITPR KO). Top: representative traces. Bottom: summary of data. Each symbol shows the maximal amplitude of Fluo-4 increase for indicated conditions. **, p<0.01 vs. vehicle (ANOVA with Dunnett's post-hoc test).

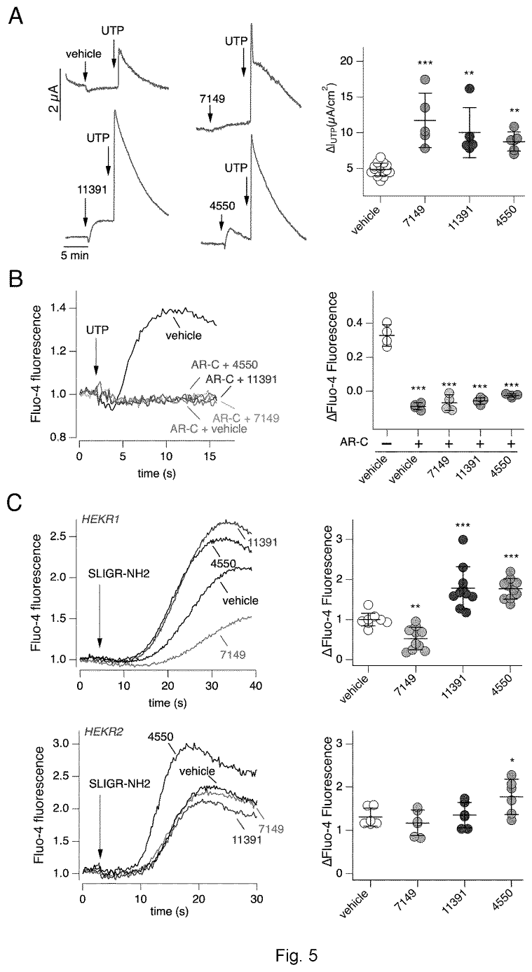

FIG. 5. Mechanism of action of ARN7149, ARN11391 and ARN4550. (A) Representative traces (left) and summary of data (right) from short-circuit current recordings on human cultured bronchial epithelia. Ca²⁺-dependent Cl⁻ secretion mediated by TMEM16A was triggered with 0.25 μM UTP on the apical side, in the presence of vehicle or indicated compounds: ARN7149 (10 μM), ARN11391 (20 μM), or ARN4550 (20 μM). To avoid the confounding effect of other channels, recordings were done in the presence of: amiloride (10 μM) to block ENaC (epithelial sodium channel); paxillin (10 μM) to block large conductance Ca²⁺-dependent K⁺ channels; inh-172 (4-[[4-Oxo-2-thioxo-3-[3-trifluoromethyl)phenyl]-5-thiazolidinylidene]methyl]benzoic acid; 10 μM, apical) to block CFTR. **, p<0.01; ***, p<0.001 vs. vehicle (ANOVA with Dunnett's post-hoc test). (B) Role of P2RY2 in mediating the effect of UTP on Ca²⁺ mobilization. Left: representative traces showing Fluo-4 fluorescence time-course in null FRT cells following extracellular addition of UTP (0.25 μM). Cells were preincubated with/without AR-C118925XX (10 μM) antagonist plus/minus ARN7149, ARN11391, or ARN4550. Right: summary of data. The symbols report the maximal change in Fluo-4 fluorescence. ***, p<0.001 vs. experiments without AR-C₁₁₈₉₂₅XX (ANOVA with Dunnett's post-hoc test). (C) Effect of compounds on Ca²⁺ mobilization triggered by SLIGR-NH₂ (protease-activate receptor agonist) in HEKR1 (top) and HEKR2 (bottom) cells. Left: representative traces showing Fluo-4 fluorescence time-course following extracellular addition of SLIGR-NH₂ (10 μM for HEKR1 and 4 μM for HEKR2). Cells were preincubated with vehicle or indicated compounds (10 μM). Right: summary of data. The symbols report the maximal change in fluorescence. *, p<0,05; **, p<0.01; ***, p<0.001 vs. vehicle (ANOVA with Dunnett's post-hoc test).

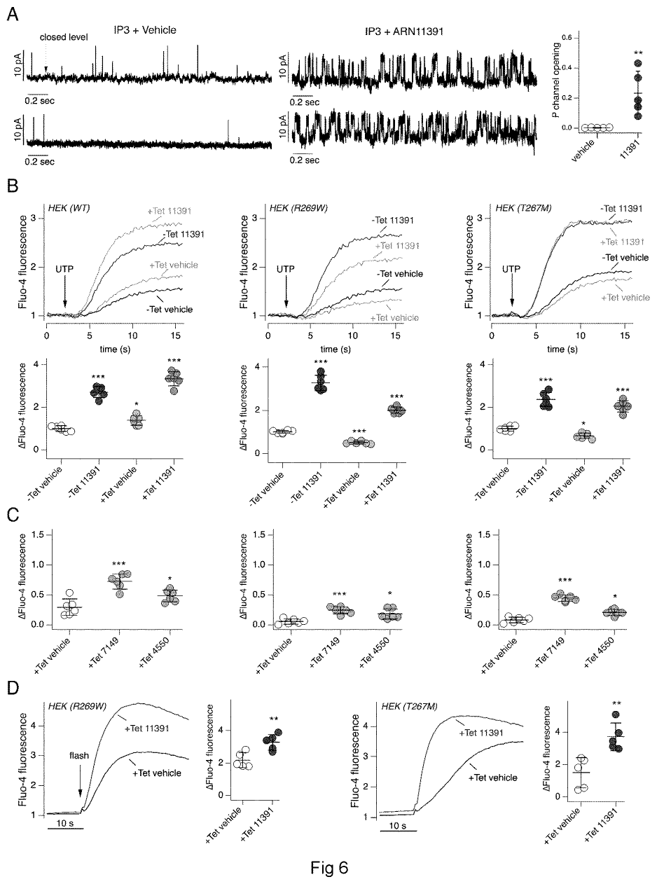

FIG. 6. Mechanism of action of ARN11391. (A) Effect of ARN11391 on ITPR1 channel activity. Left: representative single-channel currents recorded in on-nucleus patch-clamp experiments from HEK293 (HEKR1) cells stably expressing ITPR1-YFP (Vp=+40 mV). Each trace is from a separate experiment. Right: open channel probability for experiments with vehicle or ARN11391 (20 μM) in the pipette solution. **. p<0.01 vs. vehicle (Student's t test). (B) Effect of ARN11391 on HEK293 cells with inducible (tetracycline) expression of wild type or mutant ITPR1. Top: representative traces showing changes in Fluo-4 fluorescence elicited by UTP (5 μM). Cells were preincubated with vehicle (DMSO) or ARN11391 (10 μM). Where indicated, tetracycline (Tet) was added to cells to induce IPTR1 expression. Bottom: summary of data. The symbols report the maximal change in fluorescence. *, p<0.05; **, p<0.01. ***, p<0.001 vs. vehicle (ANOVA with Dunnett's post-hoc test). (C) Data from Fluo-4 experiments done on HEK293 cells expressing wild type (left), R269W (middle), and T267M (right) ITPR1. Cell were previously treated with tetracycline and then stimulated with 5 μM UTP as in (B). *, p<0.05; ***, p<0.001 (ANOVA with Dunnett's post-hoc test). (D) IP3 uncaging experiments carried out in HEK293 cells expressing the indicated mutant ITPR1. Cells were treated with tetracycline before experiments. Uncaging was done in the presence and absence of 10 μM ARN11391. **, p<0.01 (Student's t test).

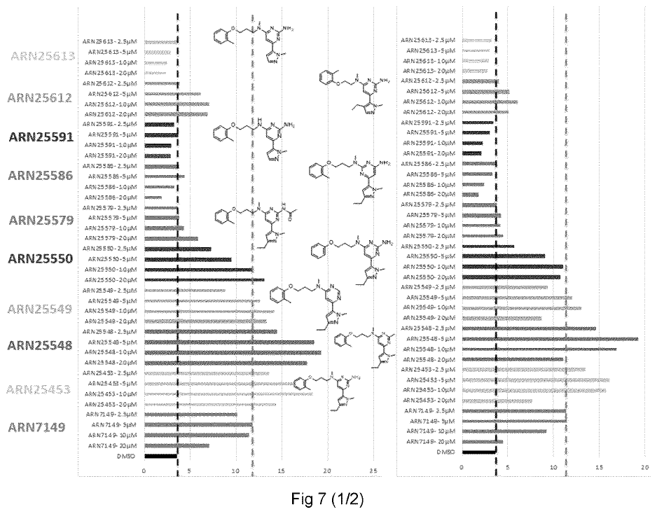

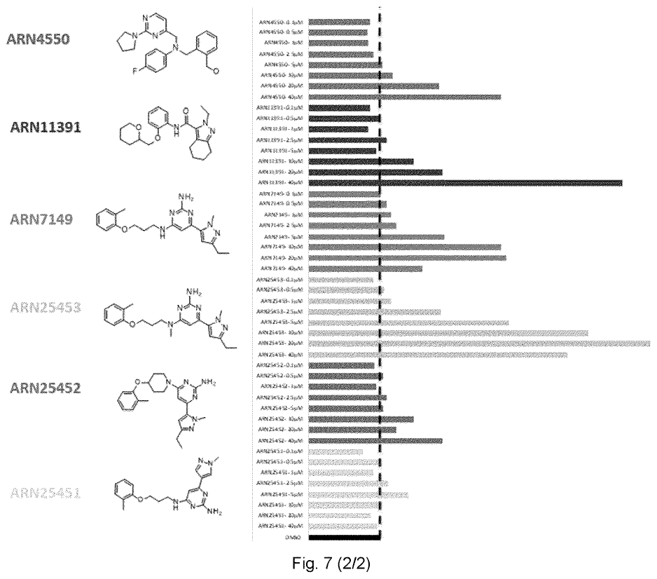

FIG. 7. Activity of the compounds. The graphs show the effect of compounds, at the indicated concentration, on anion transport in FRT cells with co-expression of the TMEM16A Cl⁻ channel and HS-YFP (12). The HS-YFP assay was done as described for FIG. 1. Data are reported as CFQ.

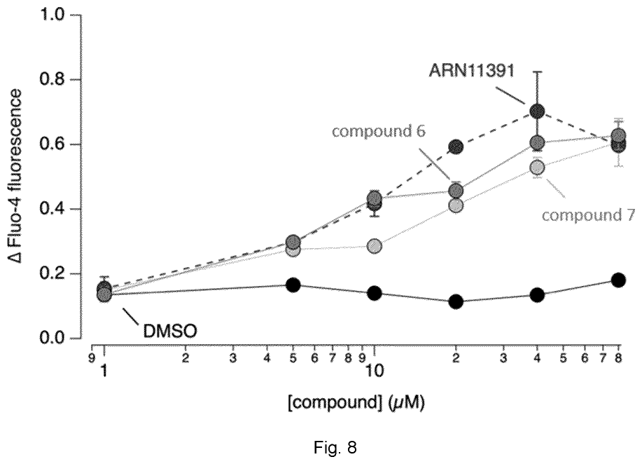

FIG. 8. Activity of ARN11391 and its analogs. The graph shows the increase in Fluo-4 fluorescence induced by UTP (0.25 μM) in FRT cells. Assays were done in the presence of indicated compounds at various concentrations or with vehicle (DMSO) alone.

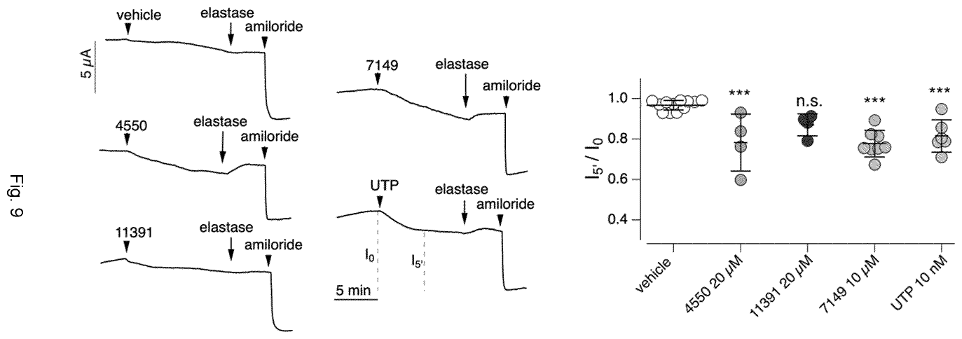

FIG. 9. Effect of compounds on ENaC function. Representative traces (left) and summary of data (right) from short-circuit current recordings on human cultured bronchial epithelia showing ENaC inhibition by direct administration of UTP (10 nM) or Ca²⁺ signaling modulators: ARN7149 (10 μM), ARN11391 (20 μM), or ARN4550 (20 μM). **, p<0.01; ***, p<0.001 vs. vehicle (ANOVA with Dunnett's post-hoc test).

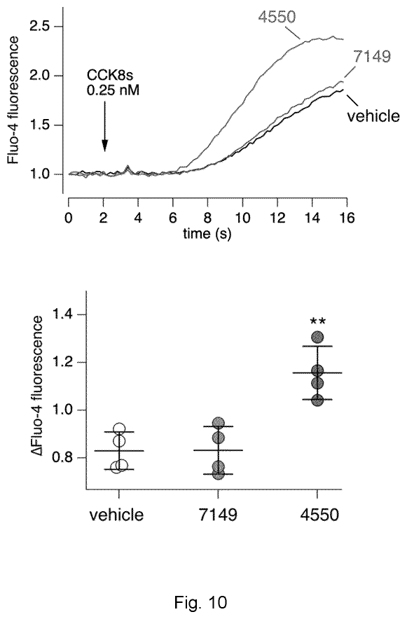

FIG. 10. Selectivity of ARN7149. Representative traces (left) and summary of data (right) showing Ca²⁺ mobilization in Chem-1 cells expressing CCK1 receptor. Cells were stimulated with CCK8s (0.25 nM) in the presence of ARN7149, ARN4550, or vehicle. The scatter dot plot reports the increase in Fluo-4 fluorescence at the end of trace. **, p<0.01 vs. vehicle (ANOVA with Dunnett's post-hoc test).

### MATERIALS AND METHODS

### Chemicals

ARN7149 (Cas #2062368-10-7; 6-(3-Ethyl-1-methyl-1H-pyrazol-5-yl)-N4-[3-(2-methylphenoxy)propyl]-2,4-pyrimidinediamine; PubChem CID: 110084502), ARN4550 (Cas #1214544-36-1; 2-[[(4-Fluorophenyl)[[2-(1-pyrrolidinyl)-4-pyrimidinyl]methyl]amino]methyl]benzenemethanol; PubChem CID: 46967215), and ARN11391 (CAS #1214569-31-9; 2H-Indazole-3-carboxamide, 2-ethyl-4,5,6,7-tetrahydro-N-[2-[(tetrahydro-2H-pyran-2-yl)methoxy]phenyl]; PubChem CID: 46969526) were purchased from AKos GmbH, Germany (catalog number: AKOS030266631, AKOS030460511, AKOS030462755, respectively); Compound 6 (CAS #1214434-83-9; 1-(2-Pyridinyl)-N-[2-[(tetrahydro-2H-pyran-2-yl)methoxy]phenyl]-3-piperidinecarboxamide) and Compound 7 (CAS 1286259-32-2; N-[2-(tetrahydropyran-2-ylmethoxy)phenyl]bicyclo[2.2.1]hept-5-ene-2-carboxamide) were purchased from Asinex (catalog n. SYN-20007833 and SYN-20118612, respectively). Compounds were dissolved as 10 mM stock solutions in DMSO. Compounds structures are indicated below:

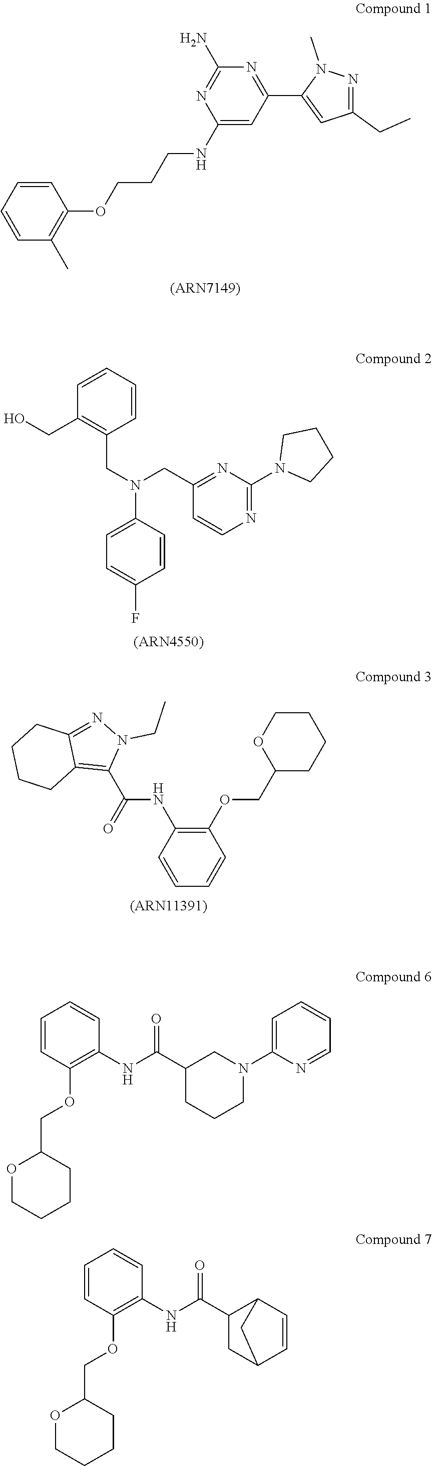 [SMILES: CCc1cc(-c2cc(NCCCOc3ccccc3C)nc(N)n2)n(C)n1 ; OCc1ccccc1CN(Cc1ccnc(N2CCCC2)n1)c1ccc(F)cc1 ; CCn1nc2c(c1C(=O)Nc1ccccc1OCC1CCCCO1)CCCC2 ; O=C(Nc1ccccc1OCC1CCCCO1)C1CCCN(c2ccccn2)C1 ; O=C(Nc1ccccc1OCC1CCCCO1)C1CC2C=CC1C2]

Non commercial compounds were prepared by standard chemistry methods according to the general synthetic procedure reported below:

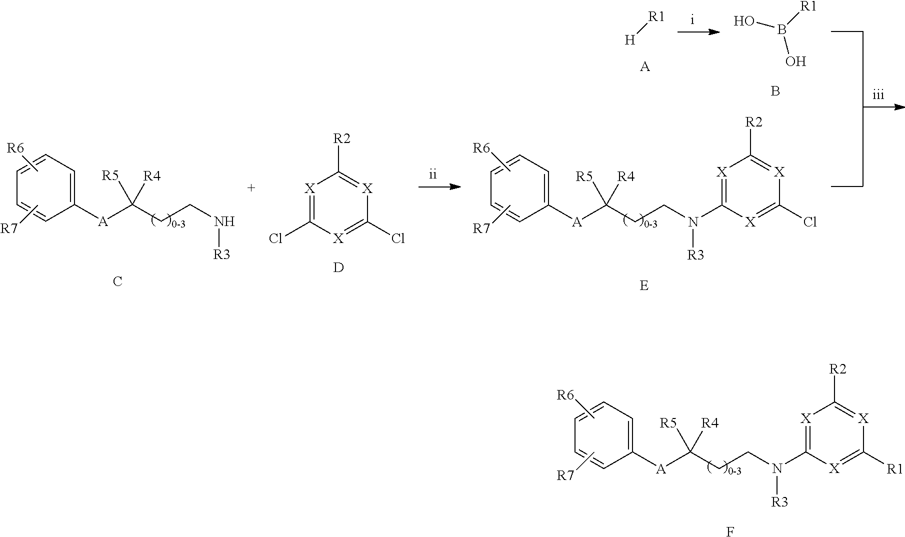

All compounds reported herein were synthesized according to the General synthetic Procedure, starting from either commercially available starting materials or in-house synthesized intermediates. A detailed description of the synthetic procedure is reported for 6-(5-ethyl-2-methyl-pyrazol-3-yl)-N⁴-methyl-N⁴-[3-(2-methylphenoxy)propyl]pyrimidine-2,4-diamine.

### 6-(5-Ethyl-2-methyl-pyrazol-3-yl)-N⁴-methyl-N⁴-[3-(2-methylphenoxy)propyl]pyrimidine-2,4-diamine (5, ARN25453)

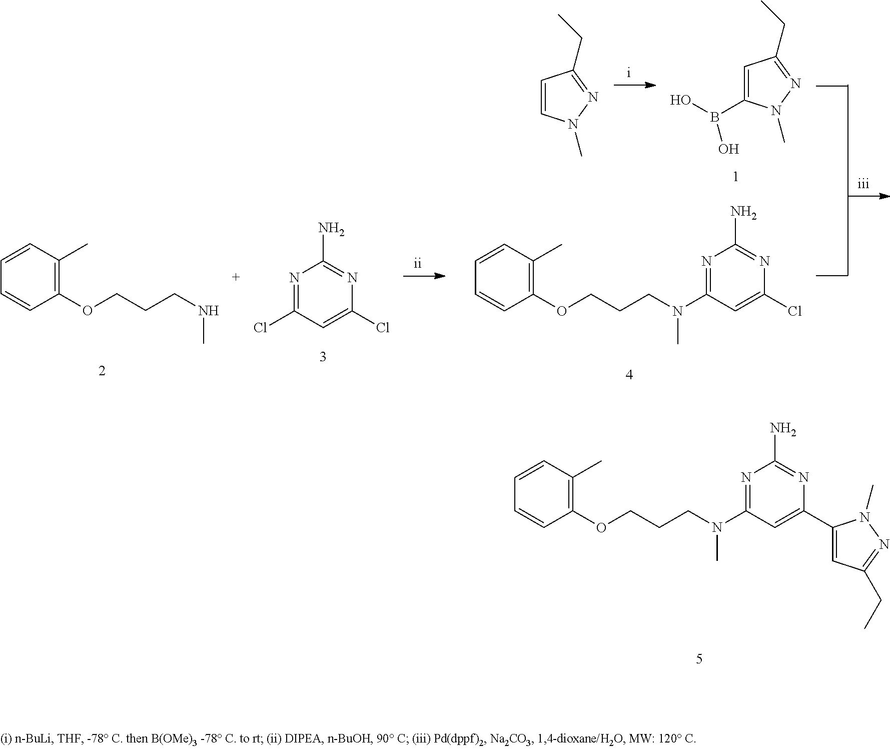

### (3-Ethyl-1-methyl-1H-pyrazol-5-yl)boronic acid (1)

3-Ethyl-1-methyl-1H-pyrazole (200 mg, 1.82 mmol) was dissolved in anhydrous THE (4 mL) and cooled to 0° C. n-Butyllithium solution (2.00 mmol, 2.5 M in hexanes) was added dropwise and reaction was allowed to reach room temperature in 2 h. The reaction was cooled down to −78° C. and trimethyl borate (2.00 mmol) was added. The solution was stirred 10 min and allowed to reach room temperature. The mixture was portioned between sat. solution of ammonium chloride and AcOEt. The organic layer was dried over anhydrous magnesium sulphate and concentrated under reduced pressure. The obtained solid was titrated with acetonitrile to obtain the title compound as an off-white solid (37% yield). ¹H NMR (400 MHz, DMSO-d₆): δ 8.24 (s, 2H), 6.50 (s, 1H), 3.88 (s, 2H), 1.20-1.08 (m, 3H).

### 6-Chloro-N-methyl-N-(3-(o-tolyloxy)propyl)pyrimidine-2,4-diamine (4)

Commercially available 3 (174 mg, 091 mmol) was dissolved in a solution of DIPEA (0.91 mmol) in n-BuOH (1.5 mL). The mixture was stirred for 10 min then commercially available 2 (150 mg, 0.91 mmol) was added. The solution was stirred for 4 h at 90° C. The solvent was removed under vacuum. After silica gel flash chromatography, eluting with a gradient from 0% to 50% of EtOAc in cyclohexane, the title compound was obtained as white solid (68% yield). UPLC/MS: Rt=2.27 min, [M+1]⁺=307.0. ¹H NMR (400 MHz, DMSO-d₆): δ 7.16-7.09 (m, 2H), 6.91-6.86 (m, 1H), 6.82 (t, J=7.3 Hz, 1H), 6.37 (s, 2H), 5.94 (s, 1H), 3.97 (t, J=6.0 Hz, 2H), 3.59 (s, 2H), 2.98 (s, 3H), 2.19 (s, 3H), 2.02-1.94 (m, 2H).

### 6-(3-Ethyl-1-methyl-1H-pyrazol-5-yl)-N-methyl-N-(3-(o-tolyloxy)propyl)pyrimidine-2,4-diamine (5; ARN25453)

6-Chloro-N-methyl-N-(3-(o-tolyloxy)propyl)pyrimidine-2,4-diamine (4) (150 mg, 0.49 mmol) and (3-ethyl-1-methyl-1H-pyrazol-5-yl)boronic acid (1) (98 mg, 0.63 mmol) were dissolved in a mixture of 1,4-dioxane (1.8 mL) and water (1.8 mL). The resulting mixture was degassed with argon for 10 min. Pd(dppf)₂ (20 mg, 0.025 mmol) and Na₂CO₃ (130 mg, 1.23 mmol) were added under argon atmosphere. The mixture was stirred at 110° C. under microwave radiation for 1 h. The crude mixture was diluted with water and then was extracted with AcOEt (2×). The organic phase was washed with brine, dried over Na₂SO₄, filtered and evaporated. After silica gel flash chromatography, eluting with a gradient from 0% to 5% of MeOH in DCM, the title compound was obtained as yellowish solid (41% yield). UPLC/MS: Rt=2.26 min, [M+1]⁺381.2. ¹H NMR (400 MHz, DMSO-d₆): δ 7.13 (t, J=6.8 Hz, 2H), 6.92-6.88 (m, J=8.2 Hz, 1H), 6.83 (t, J=7.3 Hz, 1H), 6.38 (s, 1H), 6.17 (s, 1H), 6.06 (s, 2H), 4.03 (s, 3H), 4.00 (t, J=6.0 Hz, 2H), 3.66 (t, J=6.4 Hz, 2H), 3.03 (s, 3H), 2.56-2.46 (m, 2H), 2.19 (s, 3H), 2.06-1.97 (m, 2H), 1.17 (t, J=7.6 Hz, 3H).

The remaining compounds were synthesized following the General synthetic Procedure. The ¹H NMR characterization is reported below:

### N⁴-[3-(2-Methylphenoxy)propyl]-6-(2-methylpyrazol-3-yl)pyrimidine-2,4-diamine (ARN25591)

¹H-NMR (400 MHz, DMSO-d₆): δ 8.01 (s, 1H), 7.76 (s, 1H), 7.17-7.07 (m, 2H), 6.91 (d, J=8.4 Hz, 1H), 6.82 (t, J=7.4 Hz, 1H), 6.78 (bs, 1H), 5.94 (s, 1H), 5.76 (s, 2H), 4.03 (t, J=6.2 Hz, 2H), 3.85 (s, 3H), 3.43-3.37 (m, 2H), 2.17 (s, 3H), 1.98 (p, J=6.4 Hz, 2H).

### 4-(3-Ethyl-1-methyl-1H-pyrazol-5-yl)-6-(4-(o-tolyloxy)piperidin-1-yl)pyrimidin-2-amine (ARN25452)

¹H-NMR (400 MHz, DMSO-d₆): δ 7.14 (t, J=7.0 Hz, 2H), 7.02 (d, J=8.4 Hz, 1H), 6.84 (t, J=7.4 Hz, 1H), 6.58 (s, 1H), 6.39 (s, 1H), 6.14 (s, 2H), 4.69-4.62 (m, 1H), 4.07 (s, 3H), 3.94-3.87 (m, 2H), 3.57-3.48 (m, 2H), 2.58-2.51 (m, 2H, overlapped with solvent signal), 2.16 (s, 3H), 1.99-1.90 (m, 2H), 1.69-1.53 (m, 2H), 1.18 (t, J=7.6 Hz, 3H).

### 6-(3-Ethyl-1-methyl-1H-pyrazol-5-yl)-N-methyl-N-(3-(o-tolyloxy)propyl)pyrimidin-4-amine (ARN25549)

¹H-NMR (400 MHz, DMSO-d₆): δ 8.53 (s, 1H), 7.16-7.08 (m, 2H), 6.89 (d, J=8.2 Hz, 2H), 6.82 (t, J=7.3 Hz, 1H), 6.58 (bs 1H), 4.04 (s, 3H), 4.00 (t, J=5.9 Hz, 2H), 3.78 (bs, 2H), 3.11 (s, 3H), 2.56 (m, 2H), 2.19 (s, 3H), 2.05 (p, J=6.2 Hz, 2H), 1.19 (t, J=7.6 Hz, 3H).

### 6-(3-Ethyl-1-methyl-1H-pyrazol-5-yl)-N,2-dimethyl-N-(3-(o-tolyloxy)propyl)pyrimidin-4-amine (ARN25548)

¹H-NMR (400 MHz, DMSO-d₆): δ 7.16-7.08 (m, 2H), 6.88 (d, J=8.2 Hz, 1H), 6.82 (td, J=7.3, 1.1 Hz, 1H), 6.69 (s, 1H), 6.53 (bs, 1H), 4.04 (s, 3H), 3.99 (t, J=5.8 Hz, 2H), 3.77 (bs, 2H), 3.09 (s, 3H), 2.62-2.50 (m, 2H), 2.38 (s, 3H), 2.20 (s, 3H), 2.03 (p, J=6.2 Hz, 2H), 1.18 (t, J=7.6 Hz, 3H).

### N⁴-Methyl-6-(1-methyl-1H-pyrazol-5-yl)-N⁴-(3-(o-tolyloxy)propyl)pyrimidine-2,4-diamine (ARN25613)

¹H-NMR (400 MHz, DMSO-d₆): δ 7.40 (d, J=2.0 Hz, 1H), 7.17-7.09 (m, 2H), 6.90 (d, J=8.6 Hz, 1H), 6.82 (td, J=7.3, 1.1 Hz, 1H), 6.59 (bs, 1H), 6.20 (s, 1H), 6.08 (bs, 2H), 4.10 (s, 3H), 4.00 (t, J=6.0 Hz, 2H), 3.67 (t, J=7.0 Hz, 2H), 3.04 (s, 3H), 2.18 (s, 3H), 2.02 (p, J=6.3 Hz, 2H).

### 6-(4-Ethyl-1-methyl-1H-pyrazol-5-yl)-N⁴-methyl-N⁴-(2-(o-tolyloxy)ethyl)pyrimidine-2,4-diamine (ARN25612)

¹H-NMR (400 MHz, DMSO-d₆): δ 7.18-7.08 (m, 2H), 6.93 (d, J=8.4 Hz, 1H), 6.82 (td, J=7.3, 1.1 Hz, 1H), 6.51 (s, 1H), 6.24 (bs, 1H), 6.10 (bs, 2H), 4.14 (t, J=5.4 Hz, 2H), 4.05 (s, 3H), 3.95-3.91 (m, 2H), 3.13 (s, 3H), 2.58-2.51 (m, 2H), 2.09 (s, 3H), 1.18 (t, J=7.6 Hz, 4H).

### N⁴-[3-(2-Methylphenoxy)propyl]-6-(1-methylpyrazol-4-yl)pyrimidine-2,4-diamine (ARN25451)

¹H-NMR (400 MHz, DMSO-d₆): δ 7.41 (d, J=1.9 Hz, 1H), 7.14 (t, J=7.0 Hz, 2H), 7.05 (bs, 1H), 6.92 (d, J=8.2 Hz, 1H), 6.83 (td, J=7.4, 1.1 Hz, 1H), 6.52 (s, 1H), 6.06 (bs, 2H), 6.04 (s, 1H), 4.10 (s, 3H), 4.04 (t, J=6.1 Hz, 2H), 3.49-3.41 (m, 2H), 2.17 (s, 3H), 2.00 (p, J=6.5 Hz, 2H).

### 6-(3-Ethyl-1-methyl-1H-pyrazol-5-yl)-N²-methyl-N²-(3-(o-tolyloxy)propyl)pyrimidine-2,4-diamine (ARN25586)

¹H-NMR (400 MHz, DMSO-d₆): δ 7.16-7.08 (m, 2H), 6.90 (d, J=8.1 Hz, 1H), 6.82 (td, J=7.4, 1.1 Hz, 1H), 6.42 (bs, 2H), 6.38 (s, 1H), 6.01 (s, 1H), 4.06 (s, 3H), 4.01 (t, J=6.2 Hz, 2H), 3.74 (t, J=7.1 Hz, 2H), 3.08 (s, 3H), 2.55 (m, 2H), 2.17 (s, 3H), 2.04 (p, J=6.4 Hz, 2H), 1.17 (t, J=7.6 Hz, 3H).

### N-(4-(3-Ethyl-1-methyl-1H-pyrazol-5-yl)-6-(methyl(3-(o-tolyloxy)propyl)amino)pyrimidin-2-yl)acetamide (ARN25579)

¹H-NMR (400 MHz, DMSO-d₆): δ 9.96 (s, 1H), 7.12 (t, J=6.9 Hz, 2H), 6.90 (d, J=8.4 Hz, 1H), 6.82 (t, J=7.4 Hz, 1H), 6.60 (s, 1H), 4.11 (s, 3H), 4.02 (t, J=5.8 Hz, 2H), 3.74 (bs, 2H), 3.10 (s, 3H), 2.55 (m, 2H), 2.24 (s, 3H), 2.18 (s, 3H), 2.06 (p, J=6.4 Hz, 2H), 1.18 (t, J=7.6 Hz, 3H).

### 6-(3-Ethyl-1-methyl-1H-pyrazol-5-yl)-N⁴-(3-(o-tolyloxy)propyl)pyrimidine-2,4-diamine (ARN7149)

¹H-NMR (400 MHz, DMSO-d₆): δ 7.18-7.10 (m, 2H), 7.00 (bs, 1H), 6.92 (d, J=8.3 Hz, 1H), 6.83 (td, J=7.3, 1.1 Hz, 1H), 6.31 (s, 1H), 6.02 (bs, 2H), 6.00 (s, 1H), 4.05 (t, J=6.1 Hz, 2H), 4.03 (s, 3H), 3.46-3.40 (m, 2H), 2.55-2.51 (m, 2H), 2.18 (s, 3H), 1.99 (p, J=6.4 Hz, 2H), 1.17 (t, J=7.6 Hz, 3H).

### 6-(3-Ethyl-1-methyl-1H-pyrazol-5-yl)-N⁴-methyl-N⁴-(3-phenoxypropyl)pyrimidine-2,4-diamine (ARN25550)

¹H-NMR (400 MHz, DMSO-d₆): δ 7.32-7.23 (m, 2H), 6.98-6.88 (m, 3H), 6.42 (bs, 1H), 6.18 (s, 1H), 6.06 (bs, 2H), 4.03 (s, 3H), 4.00 (t, J=6.1 Hz, 2H), 3.66-3.60 (m, 2H), 3.02 (s, 3H), 2.55-2.51 (m, 2H), 2.00 (p, J=6.4 Hz, 2H), 1.17 (t, J=7.6 Hz, 3H).

### 2-Ethyl-N-[2-(tetrahydropyran-2-ylmethoxy)phenyl]-4,5,6,7-tetrahydroindazole-3-carboxamide (ARN11391)

UPLC-MS: t_{R}=3.61 min (apolar method); [M+H]⁺: 384.4. ¹H NMR (400 MHz, DMSO-d₆): δ 9.41 (s, 1H), 8.36-8.34 (m, 1H), 7.08-6.92 (m, 3H), 4.09-4.00 (m, 4H), 3.95-3.91 (m, 1H), 3.71-3.66 (m, 1H), 3.46-3.40 (m, 1H), 2.72-2.62 (m, 4H), 1.89-1.84 (m, 1H), 1.79-1.74 (m, 3H), 1.70-1.64 (m, 2H), 1.57-1.51 (m, 4H), 1.39 (t, J=7.3 Hz, 3H).

### 1-(2-Pyridyl)-N-[2-(tetrahydropyran-2-ylmethoxy)phenyl]piperidine-3-carboxamide (compound 6)

UPLC-MS: t_{R}=4.92 min (generic method); [M+H]⁺: 396.4. ¹H NMR (400 MHz, DMSO-d₆): δ 9.00 (s, 1H), 8.12-8.10 (m, 1H), 7.87 (d, J=7.8 Hz, 1H), 7.54-7.49 (m, 1H), 7.05 (d, J=4.1 Hz, 2H), 6.95-6.86 (m, 2H), 6.62-6.60 (m, 1H), 4.39 (d, J=13.1 Hz, 1H), 4.17 (d, J=13.1 Hz, 1H), 3.97-3.84 (m, 3H), 3.65-3.58 (m, 1H), 3.44-3.35 (m, 1H), 3.05-2.98 (m, 1H), 2.92-2.85 (m, 1H), 2.68-2.60 (m, 1H), 2.01-1.95 (m, 1H), 1.80-1.63 (m, 4H), 1.55-1.26 (m, 5H).

### N-[2-(Tetrahydropyran-2-ylmethoxy)phenyl]bicyclo[2.2.1]hept-5-ene-2-carboxamide (compound 7)

UPLC-MS: t_{R}=5.15 min (generic method); [M+H]⁺: 328.1. ¹H NMR (400 MHz, DMSO-d₆): δ 8.53 (s, 1H), 7.86 (t, J=8.5 Hz, 1H), 7.06-6.98 (m, 2H), 6.92-6.87 (m, 1H), 6.20-6.18 (m, 1H), 5.98-5.94 (m, 1H), 4.01-3.91 (m, 3H), 3.70-3.63 (m, 1H), 3.46-3.40 (m, 1H), 3.26-3.22 (m, 1H), 3.13-3.08 (m, 1H), 2.91-2.87 (m, 1H), 1.91-1.81 (m, 2H), 1.69-1.64 (m, 1H), 1.56-1.46 (m, 3H), 1.42-1.31 (m, 4H).

### [2-[[4-Fluoro-N-[(2-pyrrolidin-1-ylpyrimidin-4-yl)methyl]anilino]methyl]phenyl]methanol (ARN4550)

UPLC-MS: t_{R}=5.06 min (generic method); [M+H]⁺: 393.4. ¹H NMR (400 MHz, DMSO-d₆): δ 8.23 (d, J=5.0 Hz, 1H), 7.42-7.40 (m, 1H), 7.25-7.11 (m, 3H), 6.95-6.89 (m, 2H), 6.57-6.54 (m, 2H), 6.45 (d, J=4.9 Hz, 1H), 5.14 (t, J=5.3 Hz, 1H), 4.74 (s, 2H), 4.57 (d, J=5.3 Hz, 2H), 4.47 (s, 2H), 3.45 (t, J=4.7 Hz, 4H), 1.92-1.89 (m, 4H).

### Experimental Part

¹H NMR experiments were run at 300 K on a Bruker Avance III 400 system (400.13 MHz), equipped with a BBI probe and Z-gradients. Chemical shifts for ¹H spectra were reported in parts per million (ppm), calibrating the residual non-deuterated solvent peak for the ¹H to 2.50 ppm for DMSO-d₆.

For UPLC-MS analyses, a 10 mM DMSO stock solution of test compound was prepared in DMSO-d₆ and further diluted 20-fold in CH₃CN—H₂O (1:1) for analysis. The analyses were run on an ACQUITY UPLC BEH C₁₈ column (100×2.1 mm ID, particle size 1.7 μm) with a VanGuard BEH C₁₈ pre-column (5×2.1 mm ID, particle size 1.7 μm). Generic method: a linear gradient was applied starting at 0-0.2 min: 10% B; 0.2-6.2 min: 10-90% B; 6.2-6.3 min: 90-100% B; 6.3-7.0 min: 100% B; A polar method: a linear gradient was applied starting at 0-0.2 min: 50% B; 0.2-6.2 min: 50-100% B; 6.2-7.0 min: 100% B.

### Cell Culture

Fischer rat thyroid (FRT) cells were cultured in Coon's modified Ham's F12 medium supplemented with 10% fetal bovine serum, 2 mM L-glutamine, 100 U/ml penicillin and 100 μg/ml streptomycin. FRT cells with stable coexpression of TMEM16A(abc) isoform and the halide-sensitive yellow fluorescent protein (HS-YFP) YFP-H148Q/1152L/F46L were previously described (12). FRT cells were also separately transfected to generate a stable clone expressing the PH-PLC6-GFP sensor (13).

HEK293 cells totally devoid of ITPR expression (HEK3XKO) or with selective expression of ITPR1 (HEKR1), ITPR2 (HEKR2), or ITPR3 (HEKR3), obtained by selective gene ablation, were purchased from Kerafast (catalog number: EURO30, EURO31, EURO32, EURO33) and cultured in a mixture of Dulbecco's Modified Eagle Medium (DMEM, high glucose version) and Ham's F12 (1:1) and supplemented with 10% fetal bovine serum, 2 mM L-glutamine, 100 U/ml penicillin and 100 μg/ml streptomycin.

HEK293 cells stably transfected with tetracycline-inducible expression of wild type or mutant (R269W or T267M) ITPR1, kindly provided by Prof. S. R. Wayne Chen (University of Calgary; ref. 14), were cultured in a mixture of Dulbecco's Modified Eagle Medium (DMEM, high glucose version) and Ham's F12 (1:1) and supplemented with 10% fetal bovine serum, 2 mM L-glutamine, 100 U/ml penicillin and 100 μg/ml streptomycin. Cells were selected with 0.5 mg/ml hygromycin B and induced with 1 μg/ml tetracycline.

Human bronchial epithelial cells (HBECs) were isolated and cultured as reported in a previous study (21; 40). Briefly, HBECs were cultured in flasks in a home-made serum-free medium (21). After four to five passages, cells were seeded at high density (500,000/cm²) on Snapwell porous insert (cc3801, Corning Costar). After 24 h from seeding, the basolateral medium was replaced with differentiation medium PneumaCult ALI (Stemcell Technologies), whereas the apical medium was totally removed to obtain the air-liquid interface (ALI) condition. Epithelia were kept under this condition for at least 2-3 weeks to achieve mucociliary differentiation.

Chem-1 cells expressing the CCK1 receptor (HTS184RTA, Merck) were cultured in Dulbecco's MEM/F12 (ECM0090L, EuroClone) with 10% fetal bovine serum, 2 mM L-glutamine, 100 U/ml penicillin and 100 μg/ml streptomycin.

### HS-YFP Assay and Library Screening

Experiments were performed using an automated microplate reader (FLUOstar Omega, BMG Labtech, Offenburg, Germany) equipped with syringe pumps and optical excitation/emission filters optimized for yellow fluorescent protein (YFP) fluorescence (ET500/20× and ET535/30m, respectively; Chroma Technology Corporation). FRT cells with co-expression of TMEM16A and HS-YFP were plated at high density in black wall/clear bottom 96-well microplates (3603, Corning). Each well in microplates was washed 3 times with 150 μl of phosphate-buffered saline (PBS). After washing, each well received 60 μl of PBS containing test compounds at 10 μM final concentration, or TMEM16A inhibitor Ani9 (15) (10 μM), or vehicle. After 20 min of incubation at 37° C., microplates were transferred to the plate reader for fluorescence assay. Each well was assayed individually for TMEM16A-mediated I⁻ influx by sampling cell fluorescence every 200 ms for 14 s. Two seconds after the start of reading, 165 μl of a modified PBS containing 137 mM KI, instead of NaCl, plus UTP 0.25 μM were automatically added by the reader. K⁺ was used in the injected solution to prevent changes of membrane potential due to altered activity of Ca²⁺-activated K⁺ channels. The fluorescence trace in each well was corrected by background subtraction and then normalized for the initial value measured before I⁻ addition (F0). The cumulative fluorescence quenching (CFQ) in the time interval between 3 and 13.8 s (representing integration of TMEM16A-dependent I⁻ influx) was quantified using a procedure compiled in Microsoft Excel according to the formula

$${Σ}_{i = 3}^{13.8} Δ ( Fi - F 0 ) .$$

HS-YFP assay was also done on HEK293 cells devoid of ITPR expression (HEK3XKO; Kerafast, catalog number: EURO30) after transient co-transfection with plasmids coding for TMEM16A(abc) and HS-YFP.

### Microplate Reader-Based Ca²⁺ Assay

Experiments were performed using the microplate reader described for the HS-YFP assay. FRT and HEK293 cells were cultured until confluence in 96-well microplates (3603, Corning). Cells were washed 2 times with PBS (150 μl/wash) and then loaded for 1 h at 37° C. with 5 μM Fluo-4/AM (Thermo Fisher Scientific; catalog number: F23917) in PBS containing 10 mM glucose, 0.5 mM sulfinpyrazone, and 1% fetal bovine serum. After loading, cells were washed 2 times with PBS-glucose-sulfinpyrazone (150 μl/wash) and incubated for 20 min at 37° C. with 60 μl of PBS plus 10 mM glucose and 0.5 mM sulfinpyrazone containing compounds of interest at 10 μM final concentration or vehicle. Each well was assayed individually. Fluo-4 fluorescence (500 nm excitation, 535 nm emission) was detected every 200 ms for 16-40 s. Two seconds after the start of fluorescence recording, the reader automatically injected 165 μl of a modified PBS containing 136 mM KCl instead of NaCl. This solution also contained 10 mM glucose, 0.5 mM sulfinpyrazone, and UTP or SLIGR-NH₂ (CAS #171436-38-7) at a final concentration that depended on the specific cell type. For UTP we used: 0.25 μM for FRT; 1 μM for HEKR2; 5 μM for HEK3XKO, HEKR1, and HEKR3. For SLIGR-NH₂ we used: 10 μM for HEKR1; 4 μM for HEKR2. These concentrations were chosen to elicit ˜20% of maximal effect based on UTP and SLIGR-NH₂ dose-response relationships. The lower agonist concentrations used for HEKR2 cells, compared to HEKR1 cells, are consistent with the higher affinity of ITPR2 for IP3 (16). The Fluo-4 fluorescence increase was quantified using a procedure compiled in Microsoft Excel after background subtraction and normalization for the initial value measured before UTP addition.

In a further experimental setting, experiments were performed using as above the FLUOstar Omega (BMG Labtech, Offenburg, Germany) microplate reader equipped with syringe pumps and optical excitation/emission filters optimized for yellow fluorescent protein (YFP) fluorescence (ET500/20× and ET535/30m, respectively; Chroma Technology Corporation). FRT and Chem-1 cells were cultured until confluence in 96-well microplates (cc3603, Corning). Cells were washed 2 times with PBS (150 μl/wash) and then loaded for 1 h at 37° C. with 5 μM Fluo-4/AM (Thermo Fisher Scientific; catalog number: F23917) in PBS containing 10 mM glucose, 0.5 mM sulfinpyrazone, and 1% fetal bovine serum. After loading, cells were washed 2 times with PBS-glucose-sulfinpyrazone (150 μl/wash) and incubated for 20 min at 37° C. with 60 μl of PBS plus 10 mM glucose and 0.5 mM sulfinpyrazone containing compounds of interest at 10 μM final concentration or vehicle. Each well was assayed individually. Fluo-4 fluorescence (500 nm excitation, 535 nm emission) was detected every 200 ms for 16-40 s. Two seconds after the start of fluorescence recording, the reader automatically injected 165 μl of a modified PBS containing 136 mM KCl instead of NaCl. This solution also contained, in addition to 10 mM glucose and 0.5 mM sulfinpyrazone, the following agonists: UTP (U6625, Merck) or CCK8s (23371, Cayman Chemical). The Fluo-4 fluorescence increase was quantified using a procedure compiled in Microsoft Excel after background subtraction and normalization for the initial value measured before UTP or CCK8s addition.

Intracellular Ca²⁺ Imaging with ci-IP₃/PM Uncaging Assay

FRT and HEK293 cells were cultured in μ-Plate96 microplates (Ibidi) at subconfluent condition. Cells were washed 2 times with PBS (150 μl/wash) and then loaded for 45 min at 37° C. with 1 μM ci-IP3/PM (Tocris; catalog number: 6210/10U) and 5 μM Fluo-4/AM (Thermo Fisher Scientific) in PBS containing 10 mM glucose, 0.5 mM sulfinpyrazone and 1% fetal bovine serum. After 45 min, cells were further incubated for 1 h at 37° C. with a fresh PBS solution containing Fluo-4/AM, glucose, sulfinpyrazone, serum, but no ci-IP3/PM. After the second loading, cells were washed 2 times with PBS-glucose-sulfinpyrazone (150 μl/wash) and incubated for 20 min at 37° C. with 125 μl of PBS plus 10 mM glucose and 0.5 mM sulfinpyrazone containing compounds of interest at 10 μM final concentration or vehicle. Each well was assayed individually using an inverted Olympus microscope equipped with 40×oil immersion objective, 530 nm emission filter, Lambda DG4 (Sutter Instrument Co., Novato, CA, USA) illumination system with a 490 nm excitation filter, (Olympus, Segrate, Italy), Prime cmos camera (Photometrics, Tucson, AZ, USA) and MetaFluor imaging acquisition software (Molecular Devices, Sunnyvale, CA, USA). Time-lapse experiments were carried out with 50 ms exposure time, 0.5 s interval and 1.8 min total duration. At 50 s, illumination was switched for 250 ms to full lamp power (no excitation filter) to generate an intense light flash and induce ci-IP3/PM photolysis and hence IP₃ uncaging. Analysis was performed with MetaFluor software, by quantifying Fluo-4 fluorescence in manually selected single cell region of interest (ROI). Eight cells were selected in each field to generate an average trace. After background subtraction, fluorescence traces were normalized for the initial value.

### PLC Assay

FRT cells with stable expression of PLC6-PH-GFP, generated in the laboratory of inventors, were cultured up to subconfluent condition in p-Plate96 microplates (Ibidi). Each well of a 96-well microplate was washed 3 times, leaving 125 μl of phosphate-buffered saline (PBS) containing compounds at 10 μM final concentration or vehicle. After 20 min of incubation at 37° C., microplate was moved on the stage of an inverted microscope equipped with GFP excitation/emission filters, 40×oil immersion objective (Olympus, Segrate, Italy), Lambda DG4 illumination system (Sutter Instrument Co., Novato, CA, USA), Prime cmos camera (Photometrics, Tucson, AZ, USA) and MetaMorph imaging acquisition software (Molecular Devices, Sunnyvale, CA, USA). Time-lapse experiments were carried out with 50 ms exposure time, 20 frame/s rate, and 2.4 min total duration. After 20 s and 90 s, UTP 0.25 μM and 100 μM (final concentrations) was respectively added. Analysis was performed with MetaMorph software, by quantifying GFP fluorescence cytoplasmic accumulation in manually selected single cell regions of interest (ROI). After background subtraction, fluorescence recordings were normalized for the initial value.

### Short-Circuit Current Recordings

Snapwell supports (3801, Corning) carrying FRT and differentiated HBEC epithelia were mounted in a vertical chamber resembling an Ussing system with internal fluid circulation (EM-CSYS-8, Physiologic Instruments, San Diego, CA, USA). For FRT epithelia, the apical and basolateral hemichambers were filled with solutions of different composition. The apical solution (5 ml) contained (in mM): 63 NaCl, 63 sodium gluconate, 0.38 KH₂PO₄, 2.13 K₂HPO₄, 2 CaCl₂, 1 MgSO₄, 20 Na-Hepes (pH 7.3), and 10 glucose. The basolateral solution (5 ml) instead contained (in mM): 126 KCl, 0.38 KH₂PO₄, 2.13 K₂HPO₄, 1 CaCl₂, 1 MgSO₄, 20 Na-Hepes (pH 7.3), and 10 glucose. Both solutions were continuously bubbled with air and the temperature of the solution was kept at 37° C. For HBECs, the same (bicarbonate-buffered) solution was used in both chambers. The composition of this solution was (in mM): 126 NaCl, 0.38 KH₂PO₄, 2.13 K₂HPO₄, 1 MgSO₄, 1 CaCl₂), 24 NaHCO₃, and 10 glucose. Both sides were continuously bubbled with a gas mixture containing 5% CO₂ and 95% air. The temperature of the solution was kept at 37° C. throughout the experiment.

Transepithelial voltage was short-circuited with a voltage-clamp (VCC MC8, Physiologic Instruments, San Diego, CA, USA) connected to the apical and basolateral chambers via Ag—AgCl electrodes and agar bridges (2% agar in 1 M KCl). The offset between voltage electrodes and the fluid resistance were cancelled before experiments. The resulting short-circuited current from each channel was recorded on a personal computer with the Acquire & Analize 2.3 software (Physiologic Instruments, San Diego, CA, USA).

In a further experimental setting, snapwell supports (cc3801, Corning) carrying differentiated bronchial epithelia generated in vitro were mounted in a vertical chamber resembling an Ussing system with internal fluid circulation (EM-CSYS-8, Physiologic Instruments, San Diego, CA, USA). The apical and basolateral hemichambers were filled with a solution containing (in mM): 126 NaCl, 0.38 KH₂PO₄, 2.13 K₂HPO₄, 1 MgSO₄, 1 CaCl₂), 24 NaHCO₃, and 10 glucose. Both sides were continuously bubbled with a gas mixture containing 5% CO₂ and 95% air. The temperature of the solution was kept at 37° C. throughout the experiment.

During recordings, the following molecules (in addition to ARN7149, ARN11391, and ARN4550) were added in the apical solution: elastase (SE2093002, Serva); UTP (U6625, Merck); amiloride (A7410, Merck). Transepithelial voltage was short-circuited with a voltage-clamp (VCC MC8, Physiologic Instruments, San Diego, CA, USA) connected to the apical and basolateral chambers via Ag—AgCl electrodes and agar bridges (2% agar in 1 M KCl). The offset between voltage electrodes and the fluid resistance were cancelled before experiments. The resulting short-circuited current from each channel was recorded on a personal computer with the Acquire & Analize 2.3 software (Physiologic Instruments, San Diego, CA, USA).

### Patch-Clamp Recordings

Nuclear membrane patch currents were recorded in HEKR1 cells stably transfected with a plasmid encoding human ITPR1 tagged with Enhanced Yellow Fluorescent Protein (EYFP) kindly provided by Prof. Colin W. Taylor (University of Cambridge) (17). For nuclei isolation, at least 1,000,000 HEKR1-ITPR1-EYFP cells plated in a 35 mm Petri dish were used following the protocol for patch clamp on the outer nuclear membrane (18,19). Briefly, a Duall homogenizer 20 tube (Kimble Chase 885482-0020) and a tissue grind (Kimble 885480-0020) were used. 20 strokes were applied to cells suspended in NIS-0 solution (25). NIS-O solution consisted of 40 ml of sucrose buffer (150 mM KCl, 250 mM sucrose, 1.4 mM β-mercaptoethanol, 10 mM Tris-HCl; pH 7.3 with KOH) supplemented with one tablet of complete protease inhibitor cocktail (Roche) and 200 μM PMSF.

Borosilicate glass pipettes were pulled on a two-step vertical puller (Narishige) to a final resistance of around 15-20 MO, as measured in the working solution. The bath solution had the following composition (in mM): 140 KCl, 0.06 CaCl₂), 0.5 EGTA, 10 K-Hepes (pH 7.3; free Ca²⁺ concentration: 70 nM). The pipette solution contained (in mM): 140 KCl, 0.5 EGTA, 0.46 CaCl₂), 10 K-Hepes, 0.5 ATP (pH 7.3), 0.008 IP₃. The pipette solution also contained 20 μM ARN11391 or DMSO.

For patch-clamp experiments, 50 μl of the suspension containing nuclei and intact cells were delivered to a 35 mm Petri dish, containing 2 ml of bath solution, and mounted on the stage of an inverted microscope. For each experiment, an isolated nucleus was chosen, using the criteria described by Mak and coll. (18,19), to attempt giga-seal formation (on-nucleus patch-clamp configuration, equivalent to cell-attached patch-clamp).

During experiments, the membrane capacitance was analogically compensated using the circuitry provided by the EPC10 patch-clamp amplifier. Experiments were performed at room temperature (20-22° C.). During recordings, the voltage pipette (Vp) was held at 0 mV with respect to the bath electrode and with 5 s-long voltage steps to Vp=+40 mV were applied with interval time of 1 s. Membrane currents were filtered at 1 kHz and digitized at 10 kHz. Data were analysed using the Igor software (WaveMetrics, Lake Oswego, OR, USA) supplemented by custom software kindly provided by Dr. Oscar Moran (Institute of Biophysics, CNR, Genova, Italy).

### Data Visualization and Statistical Analysis

Data are shown as representative images/traces and as scatter dot plots with mean±SD. Each symbol in the plots represents the result of an independent experiment. To assess significant differences between groups of data, we used ANOVA followed by Dunnett's or Tukey's post hoc tests as appropriate. Statistical analysis was done with PRISM software (GraphPad). All graphs and figures were prepared with Igor Pro (WaveMetrics).

### Results

### Identification of Active Compounds by High-Throughput Screening

Fischer Rat Thyroid (FRT) cells generated by stable transfection with TMEM16A and HS-YFP plasmids (12) were used. FRT cells are a convenient cell model since they do not express other anion channels and transporters. Furthermore, they form tight epithelia with high electrical resistance when plated on porous membrane (20). In this way, transepithelial ion transport in FRT cells can be studied with the short-circuit current technique.

For the screening, FRT cells were plated at high density in 96-well microplates. After 48 h, TMEM16A activity was evaluated in a microplate reader by injection in each well of a saline solution containing I⁻ instead of Cl⁻ and a submaximal (0.25 μM) UTP concentration (FIG. 1A). Upon injection, UTP triggers Ca²⁺ mobilization and TMEM16A activation. The resulting TMEM16A-dependent I⁻ influx causes HS-YFP quenching. The presence in the well of a small molecule that potentiates TMEM16A activity, in a direct or indirect way, is therefore expected to enhance the rate of HS-YFP quenching.

It was screened a chemical library of 11,300 compounds, generated at the Italian Institute of Technology, and having maximal structural diversity and good drug-like properties. This library was previously used to find CFTR correctors and potentiators (21,22). All compounds were tested at 10 μM by addition in the microplate 20 minutes before the assay. Control wells in each microplate included vehicle alone or TMEM16A inhibitor Ani9 (15). Primary hits, showing potentiation of TMEM16A activity, were retested in the HS-YFP assay to confirm activity (FIG. 1B). Compounds passing this test were further evaluated in short-circuit current recordings on FRT epithelia. As shown in FIG. 1C, the peak of Cl⁻ current elicited by UTP was significantly enhanced by three compounds: ARN7149, ARN11391, and ARN4550. The lack of effect of other two compounds, ARN12881 and ARN4360, in the short-circuit current assay could imply that they act on an endogenous electroneutral anion transporter and not on TMEM16A. We decided to continue the characterization of ARN7149, ARN11393, and ARN4550. The dose-response relationships was determined for these three compounds testing multiple concentrations with the HS-YFP assay (FIG. 1D). ARN11391 and ARN4550 were effective at concentrations 10 μM. ARN7149 had instead a bell-shaped dose-response relationship with activity in the 5-20 μM range.

### Mechanism of Action of Active Compounds on Intracellular Ca²⁺ Mobilization

Intracellular Ca²⁺ mobilization was monitored with the Fluo-4 Ca²⁺-sensitive fluorescent probe. For these experiments, parental FRT cells devoid of TMEM16A expression were used. FIG. 2A shows that ARN7149, ARN11391, and ARN4550 were all able to potentiate UTP-dependent Ca²⁺ increase. These results indicated that the three compounds act on TMEM16A with an indirect mechanism of action. Subsequently, we determined whether these compounds were able to directly mobilize Ca²⁺ in the absence of UTP. Compounds were injected during recording of Fluo-4 fluorescence (FIG. 2B). No effect was found in contrast to Eact, a compound that elicits Ca²⁺ influx by activating TRPV4 channel (23). These results indicated that ARN7149, ARN11391, and ARN4550 act as potentiators on components of the Ca²⁺ signaling cascade and not Ca²⁺-elevating agents by themselves.

To elucidate the mechanism of action of active compounds identified in the screening, a series of experiments were carried out. Since Ca²⁺ release mediated by ITPR opening is a key step in the Ca²⁺ signaling cascade, the first set of experiments was done on HEK293 cells in which selective ablation of endogenous ITPR genes was obtained by gene editing (24,25). Ca²⁺ mobilization elicited by UTP was evaluated with the Fluo-4 probe (FIG. 2C). Interestingly, ARN7149 and ARN4550 were always effective in potentiating the Ca²⁺ increase irrespective of ITPR type expression. In contrast, ARN11391 was only effective in cells with ITPR1 as the only ITPR type (FIG. 2C). As expected, no Ca²⁺ mobilization by UTP plus/minus potentiator was observed in HEK293 cells completely devoid of ITPRs (FIG. 2C). The lack of effect in ITPR-defective cells was also investigated using TMEM16A as a reporter. For this purpose, HEK293 cells without ITPR1-3 expression were transiently transfected with plasmids coding for TMEM16A and HS-YFP. In agreement with the lack of Ca²⁺ mobilization, the HS-YFP assay showed no activation of TMEM16A by UTP with/without ARN11391 (FIG. 2D), Instead, a large effect was observed when Ca²⁺ was directly increased by ionomycin (FIG. 2D).

The second type of experiments was done with an assay developed to monitor PLC activity. FRT cells with stable expression of the PH-PLC6-GFP fluorescent sensor were generated. Under resting conditions, the pleckstrin homology (PH) domain, which binds to PIP₂, anchors the sensor to the inner side of the membrane (FIG. 3A). Upon activation of PLC by UTP, PIP₂ hydrolysis releases PH-PLC6-GFP, which then redistributes to the cytosol. UTP was sequentially added at two concentrations (0.25 and 100 μM), in the presence/absence of potentiators, and the increase in GFP fluorescence was measured in the cytosol as the parameter reflecting PLC activation (FIG. 3A). With the lower UTP concentration, ARN7149 and ARN4550, but not ARN11391, significantly potentiated UTP effect. With the higher UTP concentration, all three compounds were effective. It is known that phospholipase C is a Ca²⁺-activated enzyme. Consequently, PLC activity can be amplified through a positive feedback loop based on Ca²⁺ release through ITPRs (26). For this reason, parallel experiments with the membrane-permeable Ca²⁺ chelator BAPTA/AM were carried out. With this compound, PLC activity, with/without compounds, was markedly inhibited (FIG. 3B).

Results shown in FIG. 2C led to hypothesize that ARN 1391 directly acts on ITPR1. Therefore, experiments with a membrane-permeable caged-IP3 (cilP3/AM) were carried out. Upon cell loading, an intense flash of light was applied to cause photolysis and release of free IP₃ (FIG. 4A). In this way, Ca²⁺ mobilization could be induced by directly activating ITPRs thus bypassing purinergic receptors and PLC. With this assay, done in FRT cells, only ARN 1391 showed activity (FIG. 4A). The caged-IP3 experiments were also carried out in HEK293 cells with selective expression of ITPR1 (HEKR1), IPTR2 (HEKR2), ITPR3 (HEKR3) or no ITPR at all (HEK3XKO). Of the three potentiators, ARN 1391 was uniquely effective in cells with selective expression of ITPR1 (FIG. 4B).

To further evaluate the activity of potentiators on purinergic signaling, the compounds were tested in native airway epithelia, in which UTP is able to activate Ca²⁺-dependent Cl⁻ secretion (27, 28). FIG. 5A shows that the transepithelial Cl⁻ current elicited by apical UTP application was significantly enhanced by ARN7149, ARN 1391, and ARN4550.

To investigate whether the potentiation of UTP stimulus involves P2RY2 or another type of purinergic receptor, AR-C₁₁₈₉₂₅XX was used as a selective P2RY2 antagonist (29). This compound completely blocked the effect of UTP alone as well that of UTP plus potentiators (FIG. 5B, compare with FIG. 2A).

In addition it was tested the activity of potentiators on other types of stimuli inducing GPCR-PLC-ITPR cascade. To this end, instead of UTP, it was used SLIGR-NH₂ that is an agonist of protease-activated receptors (30). These experiments were done on HEK293 cells expressing ITPR1 (HEKR1) or ITPR2 (HEKR2). Interestingly, ARN11391 and ARN4550, but not ARN7149, were effective as potentiators in HEKR1 cells (FIG. 5C, top). Actually, ARN7149 caused a significant inhibition in this set of experiments. In HEKR2 cells, ARN4550 was the only compound that potentiated the SLIGR-NH₂ stimulus (FIG. 5C, bottom). These results indicate that ARN7149 may be used to potentiate the Ca²⁺ signaling cascade by selectively acting on the P2RY2 receptor.

### ARN11391 as an ITPR1 Potentiator

It appears particularly interesting the possibility of ARN11391 being a direct ITPR1 potentiator, also given that no known selective activators/potentiators of ITPRs in general seem to be known in the scientific literature. Therefore, cells overexpressing ITPR1 were used to carry out nuclear patch-clamp recordings (18,19). In these experiments, the outer nuclear membrane serves as a surrogate of endoplasmic reticulum thus allowing recording of ITPR single channel activity (18,19). Experiments were done with/without ARN11391 in the pipette solutions (FIG. 6A). It was observed single channel openings of the expected current amplitude for ITPR1 (˜10 pA at +40 mV) in 5 out of 40 attempts with vehicle alone, and in 5 out of 32 attempts with ARN11391. In the absence of compound (vehicle alone), openings were rare and open channel probability (Po) was well below 0.01. With ARN11391, recordings showed much higher channel activity, with an average Po value close to 0.2 (FIG. 6A).

ITPR1 gene may be affected by loss-of-function mutations in some forms of spinocerebellar ataxia (SCA29) (31). The inventors investigated whether ARN11391 is effective in cells expressing mutant ITPR1. For this, the compound was tested on two SCA29 mutations: R269W and T267M. Experiments were done on cells with inducible expression of these ITPR1 mutants (14). Cells were pre-treated with/without tetracycline and then recorded the intracellular Ca²⁺ increase elicited by UTP. Results were compared with those of cells expressing wild type ITPR1. Induction had opposite effects depending on the cell type: an increase in signal for wild type ITPR1 and a significant decrease for R269W- and T267M-ITPR1 (FIG. 6B). This decrease can be interpreted as the dominant negative effect caused by overexpression of mutant ITPR1 over endogenous ITPRs. Importantly, ARN11391 elicited a marked potentiation of Ca²⁺ mobilization in cells with R269W and T267M mutants (FIG. 6B). For comparison, ARN7149 and ARN4550 were also tested in cells expressing mutant ITPR1 (FIG. 6C). These compounds were also effective although less than ARN11391. The efficacy of ARN11391 on mutant ITPR1 was also evaluated with the caged IP₃ assay. In cells expressing R269W- and T267M-ITPR1, ARN11391 significantly amplified the Ca²⁺ mobilization elicited by IP3 uncaging (FIG. 6D).

### Activity of ARN11391 Analogs

Chemical analogs of ARN11391 (labeled as compound 6 and compound 7) were tested in the Ca²⁺ mobilization assay to assess their activity. FRT cells were loaded with the Ca²⁺-sensitive Fluo-4 probe (39) and then stimulated with UTP (0.25 μM) in the presence of ARN11391, compound 1, and compound 2 at multiple concentrations in the range 1-80 μM. All three compounds caused a dose-dependent potentiation of the Ca²⁺ increase elicited by UTP compared to vehicle (DMSO) alone (see FIG. 8).

### Effect of Potentiators on ENaC Function

The properties of the airway surface are also modulated by the activity of the epithelial Na⁺ channel ENaC (43). Na⁺ absorption through ENaC causes airway surface dehydration and is therefore detrimental for mucociliary clearance. For this reason, ENaC is considered a possible pharmacological target to treat cystic fibrosis and other chronic obstructive pulmonary diseases. Inhibition of ENaC is expected to improve airway surface hydration.

It was investigated whether compounds of the invention, besides enhancing TMEM16A-dependent Cl⁻ secretion, were also able to inhibit ENaC. The basis for this hypothesis is the known dependence of ENaC activity on the levels of phosphatidylinositol 4,5-bisphosphate, PIP2 (42). More precisely, PIP2 breakdown upon phospholipase C activation leads to ENaC inhibition. It was therefore studied whether the potentiators of Ca²⁺ signaling cascades have also an effect on ENaC. The inventors carried out experiments in which ARN7149, ARN4550, and ARN11391 were added on the apical membrane of epithelia during short-circuit current recordings. Airway epithelia tonically release ATP as an autocrine mechanism involving P2Y2 receptors (41). The underlying hypothesis was that addition of compounds, particularly ARN7149 and ARN4550, should potentiate the PIP2 breakdown caused by endogenous ATP release, thus leading to ENaC inhibition. Indeed, ARN7149 and ANR4550, but not ARN11391 which acts downstream phospholipase C, caused ENaC current decay similarly to that elicited by addition of UTP (FIG. 9).

### Selectivity of ARN7149

To evaluate the selectivity of ARN1749 as a potentiator of P2RY2, the inventors tested this compound on a commercially available (Merck) cell line, Chem-1, with expression of the CCK1 colecystokinin receptor (FIG. 10). Cells were loaded with the Ca²⁺-sensitive probe Fluo-4 and stimulated with a CCK1 agonist (CCK8s, 0.25 nM) in the presence of ARN7149, ARN4550, or vehicle (DMSO). The mobilization of intracellular Ca²⁺ was measured with a microplate reader as previously described (39). ARN4550, but not ARN7419, significantly enhanced CCK8s effect. The lack of activity of ARN7149 is consistent with this compound being a selective P2RY2 receptor modulator. Indeed, ARN7149 is only effective when Ca²⁺ mobilization is induced by UTP and not by other agonists of G-protein coupled receptors (GPCR) such as protease-activated and bradykinin receptors (39). On the other hand, the activity of ARN4550 in the CCK1 assay further supports the conclusion that this compound acts on a common step downstream GPCRs, possibly phospholipase C.

### DISCUSSION

In the present invention, the activity of the Ca²⁺-activated TMEM16A Cl⁻ channel was used as the functional readout to identify TMEM16A potentiators as well as modulators of the Ca²⁺ signaling cascade. After the primary screening of a chemical library and secondary tests, were identified three compounds, ARN7149, ARN11391, and ARN4550, that significantly potentiated the effect of UTP on TMEM16A. The mechanism of action of the compounds was assessed by applying a series of functional assays. For two of them, ARN11391 and ARN7149, convincing evidence was found indicating ITPR1 and P2RY2 as the probable targets, respectively.

Regarding ARN 1391, the compound is effective in the caged IP₃ assay in which all other upstream steps (membrane receptors, PLC) are bypassed. Furthermore, ARN 1391 is only effective when ITPR1 is expressed. The inventors also carried out nuclear patch-clamp experiments on ITPR1-expressing cells that demonstrated a marked increase in channel activity when ARN11391 was included (FIG. 6A). Such results are supportive of a mechanism involving direct interaction of the compound with ITPR1 protein. Intriguingly, in the PLC assay, ARN 1391 was ineffective with the submaximal UTP stimulus, but effective with the maximal UTP stimulus. This latter result could appear inconsistent with a mechanism based on ITPR1 binding since PLC is localized upstream in the signaling cascade. However, the effect of ARN11391 on PLC can be explained with a positive feedback loop connecting ITPR activation and Ca²⁺-dependent PLC (26). Indeed, this link is demonstrated by showing that PLC activity can be markedly inhibited with the BAPTA Ca²⁺-chelating agent. Interestingly, ARN11391 was effective in cells expressing ITPR1 with mutations causing SCA29. Such results indicate the therapeutic application of ARN 1391-like compounds in patients with spinocerebellar ataxia caused by ITPR1 defective function. ARN7149 and ARN4550 showed some activity on ITPR1 mutants (FIG. 6C). In this respect, R269W and T267M have been shown to decrease affinity of ITPR1 for IP₃ (14), probably by affecting the IP₃ binding site (31). Therefore, the effect of ARN7149 and ARN4550 can be explained with an indirect mechanism on ITPR1, due to enhanced IP₃ concentration that overcomes the decreased affinity of the mutant receptor for IP₃.

ARN7149 was the other compound for which were obtained indications on the possible mechanism of action. This compound was effective in the PLC assay. It was also effective in the Ca²⁺ mobilization assay irrespective of expression of a particular ITPR type. Finally, it was inactive in the caged IP₃ assay (FIG. 4) thus ruling out ITPRs as the target. All these results place ARN7149 site of action on an early step of the GPCR-PLC-ITPR cascade. Lack of effect on other stimuli, involving other receptors, indicates that P2RY2 is the target of ARN7149, as also indicated by results with the AR-C 118925XX antagonist (FIG. 5). Results obtained with the antagonist indicate that P2RY2, and not another purinergic receptor, is involved in ARN7149 activity. Potentiators of P2RY2 receptors could be potentially useful as therapeutic agents for a series of human diseases (32). In particular, they can be used topically to improve fluid and mucin secretion in dry eye syndrome (33,34). A non-nucleotide agonist of P2RY2 attenuated isoprotenerol-induced cardiomyocyte hypertrophy (35). Potentiation of P2RY2 could also be useful to promote Ca²⁺ -dependent Cl⁻ secretion in airway epithelia. In this respect, there is evidence of a tonic release of ATP by airway epithelia that is further enhanced by mechanical stress (36-38). Released ATP then acts in an autocrine way on epithelial cells activating TMEM16A through P2RY2. Therefore, ARN7149-like compounds could enhance Cl⁻ secretion and airway surface hydration, an effect that could be beneficial in CF and other chronic obstructive respiratory diseases. However, intracellular Ca²⁺ elevation could also promote mucus secretion.

Regarding ARN4550, the precise site of action was not identified. This compound does not act on ITPRs since it was inactive in IP₃ uncaging experiments. Also, it does not act on a specific GPCR, since it potentiated the Ca²⁺ mobilization by both UTP and SLIGR-NH2 stimuli. Therefore, ARN4550 may act on a step intermediate between GPCR and PLC, possibly a G protein, the PLC itself, or another related regulatory protein.

In conclusion, the present invention used a functional screening assay to identify novel modulators of the Ca²⁺ signaling cascade. Such compounds are important as mechanistic probes for scientific research purposes and as novel therapeutic agents.

### REFERENCES

1. Berridge M J. The inositol trisphosphate/calcium signaling pathway in health and disease. Physiol Rev 96:1261-1296, 2016.
2. Prole D L, Taylor C W. Inositol 1,4,5-trisphosphate receptors and their protein partners as signalling hubs. J Physiol 594:2849-2866, 2016.
3. Taylor C W, Genazzani A A, Morris S A. Expression of inositol trisphosphate receptors. Cell Calcium 26:237-251, 1999.
4. Foskett J K, White C, Cheung K H, Mak D O. Inositol trisphosphate receptor Ca²⁺ release channels. Physiol Rev 87:593-658, 2007.
5. Brini M, Cali T, Ottolini D, Carafoli E. Neuronal calcium signaling: function and dysfunction. Cell Mol Life Sci 71:2787-2814, 2014.
6. Huang F, Rock J R, Harfe B D, Cheng T, Huang X, Jan Y N, Jan L Y. Studies on expression and function of the TMEM16A calcium-activated chloride channel. Proc Natl Acad Sci USA 106:21413-21418, 2009.
7. Twyffels L, Strickaert A, Virreira M, Massart C, Van Sande J, Wauquier C, Beauwens R, Dumont J E, Galietta L J, Boom A, Kruys V. Anoctamin-1/TMEM16A is the major apical iodide channel of the thyrocyte. Am J Physiol 307:C1102-C1112, 2014.
8. Dutta A K, Boggs K, Khimji A K, Getachew Y, Wang Y, Kresge C, Rockey D C, Feranchak A P. Signaling through the interleukin-4 and interleukin-13 receptor complexes regulates cholangiocyte TMEM16A expression and biliary secretion. Am J Physiol 318:G763-G771, 2020.
9. Oh U, Jung J. Cellular functions of TMEM16/anoctamin. Pflugers Arch 468:443-453, 2016.
10. Galietta L J V. TMEM16A (ANO1) as a therapeutic target in cystic fibrosis. Curr Opin Pharmacol 64:102206, 2022.
11. Galietta L J, Haggie P M, Verkman A S. Green fluorescent protein-based halide indicators with improved chloride and iodide affinities. FEBS Lett 499:220-224, 2001.
12. Caputo A, Caci E, Ferrera L, Pedemonte N, Barsanti C, Sondo E, Pfeffer U, Ravazzolo R, Zegarra-Moran O, Galietta U. TMEM16A, a membrane protein associated with calcium-dependent chloride channel activity. Science 322:590-594, 2008.
13. Szentpetery Z, Balla A, Kim Y J, Lemmon M A, Balla T. Live cell imaging with protein domains capable of recognizing phosphatidylinositol 4,5-bisphosphate; a comparative study. BMC Cell Biol 10:67, 2009
14. Synofzik M, Helbig K L, Harmuth F, Deconinck T, Tanpaiboon P, Sun B, Guo W, Wang R, Palmaer E, Tang S, Schaefer G B, Gburek-Augustat J, Zuchner S, Krageloh-Mann I, Baets J, de Jonghe P, Bauer P, Chen S R W, Schöls L, Schule R. De novo ITPR1 variants are a recurrent cause of early-onset ataxia, acting via loss of channel function. Eur J Hum Genet 26:1623-1634, 2018.
15. Seo Y, Lee H K, Park J, Jeon D K, Jo S, Jo M, Namkung W. Ani9, a novel potent small-molecule ANO1 inhibitor with negligible effect on ANO2. PLoS One 11:e0155771, 2016.
16. Iwai M, Tateishi Y, Hattori M, Mizutani A, Nakamura T, Futatsugi A, Inoue T, Furuichi T, Michikawa T, Mikoshiba K. Molecular cloning of mouse type 2 and type 3 inositol 1,4,5-trisphosphate receptors and identification of a novel type 2 receptor splice variant. J Biol Chem 280:10305-10317, 2005.
17. Pantazaka E, Taylor C W. Targeting of inositol 1,4,5-trisphosphate receptor to the endoplasmic reticulum by its first transmembrane domain. Biochem J 425:61-69, 2009.
18. Mak D O, Vais H, Cheung K H, Foskett J K. Isolating nuclei from cultured cells for patch-clamp electrophysiology of intracellular Ca²⁺ channels. Cold Spring Harb Protoc 2013:880-884, 2013.
19. Mak D O, Vais H, Cheung K H, Foskett J K. Patch-clamp electrophysiology of intracellular Ca²⁺ channels. Cold Spring Harb Protoc 2013:787-797, 2013.
20. Sheppard D N, Carson M R, Ostedgaard L S, Denning G M, Welsh M J. Expression of cystic fibrosis transmembrane conductance regulator in a model epithelium. Am J Physiol 266:L405-L413, 1994.
21. Pedemonte N, Bertozzi F, Caci E, Sorana F, Di Fruscia P, Tomati V, Ferrera L, Rodriguez-Gimeno A, Berti F, Pesce E, Sondo E, Gianotti A, Scudieri P, Bandiera T, Galietta L J V. Discovery of a picomolar potency pharmacological corrector of the mutant CFTR chloride channel. Sci Adv 6:eaay9669, 2020.
22. Brindani N, Gianotti A, Giovani S, Giacomina F, Di Fruscia P, Sorana F, Bertozzi S M, Ottonello G, Goldoni L, Penna I, Russo D, Summa M, Bertorelli R, Ferrera L, Pesce E, Sondo E, Galietta L J V, Bandiera T, Pedemonte N, Bertozzi F. Identification, structure-activity relationship, and biological characterization of 2,3,4,5-tetrahydro-1H-pyrido[4,3-b]indoles as a novel class of CFTR potentiators. J Med Chem 63:11169-11194, 2020.
23. Genovese M, Borrelli A, Venturini A, Guidone D, Caci E, Viscido G, Gambardella G, di Bernardo D, Scudieri P, Galietta L J V. TRPV4 and purinergic receptor signalling pathways are separately linked in airway epithelia to CFTR and TMEM16A chloride channels. J Physiol 597:5859-5878, 2019.
24. Alzayady K J, Wang L, Chandrasekhar R, Wagner L E 2nd, Van Petegem F, Yule D I. Defining the stoichiometry of inositol 1,4,5-trisphosphate binding required to initiate Ca²⁺ release. Sci Signal 9:ra35, 2016.
25. Mataragka S, Taylor C W. All three IP3 receptor subtypes generate Ca²⁺ puffs, the universal building blocks of IP3-evoked Ca²⁺ signals. J Cell Sci 131:jcs220848, 2018.
26. Horowitz L F, Hirdes W, Suh B C, Hilgemann D W, Mackie K, Hille B. Phospholipase C in living cells: activation, inhibition, Ca²⁺ requirement, and regulation of M current. J Gen Physiol 126:243-262, 2005.
27. Scudieri P, Caci E, Bruno S, Ferrera L, Schiavon M, Sondo E, Tomati V, Gianotti A, Zegarra-Moran O, Pedemonte N, Rea F, Ravazzolo R, Galietta L J. Association of TMEM16A chloride channel overexpression with airway goblet cell metaplasia. J Physiol 590:6141-6155, 2012
28. Gorrieri G, Scudieri P, Caci E, Schiavon M, Tomati V, Sirci F, Napolitano F, Carrella D, Gianotti A, Musante I, Favia M, Casavola V, Guerra L, Rea F, Ravazzolo R, Di Bernardo D, Galietta U. Goblet cell hyperplasia requires high bicarbonate transport to support mucin release. Sci Rep 6:36016, 2016.
29. Muoboghare M O, Drummond R M, Kennedy C. Characterisation of P2Y2 receptors in human vascular endothelial cells using AR-C118925XX, a competitive and selective P2Y2 antagonist. Br J Pharmacol 176:2894-2904, 2019.
30. Hollenberg M D, Saifeddine M, al-Ani B. Proteinase-activated receptor-2 in rat aorta: structural requirements for agonist activity of receptor-activating peptides. Mol Pharmacol 49:229-233, 1996.
31. Ando H, Hirose M, Mikoshiba K. Aberrant IP3 receptor activities revealed by comprehensive analysis of pathological mutations causing spinocerebellar ataxia 29. Proc Natl Acad Sci USA 115:12259-12264, 2018.
32. Burnstock G. Purinergic signalling: therapeutic developments. Front Pharmacol 8:661, 2017.
33. Jumblatt J E, Jumblatt M M. Regulation of ocular mucin secretion by P2Y2 nucleotide receptors in rabbit and human conjunctiva. Exp Eye Res 67:341-346, 1998.
34. Lau O C, Samarawickrama C, Skalicky S E. P2Y2 receptor agonists for the treatment of dry eye disease: a review. Clin Ophthalmol 8:327-334, 2014.
35. Sakuma K, Nakagawa H, Oikawa T, Noda M, Ikeda S. Effects of 4(1H)-quinolinone derivative, a novel non-nucleotide allosteric purinergic P2Y2 agonist, on cardiomyocytes in neonatal rats. Sci Rep 7:6050, 2017.
36. Okada S F, Nicholas R A, Kreda S M, Lazarowski E R, Boucher R C. Physiological regulation of ATP release at the apical surface of human airway epithelia. J Biol Chem 281:22992-23002, 2006.
37. Button B, Picher M, Boucher R C. Differential effects of cyclic and constant stress on ATP release and mucociliary transport by human airway epithelia. J Physiol 580:577-592, 2007.
38. Button B, Okada S F, Frederick C B, Thelin W R, Boucher R C. Mechanosensitive ATP release maintains proper mucus hydration of airways. Sci Signal 6:ra46, 2013.
39. Genovese M, Guidone D, Buccirossi M, Borrelli A, Rodriguez-Gimeno A, Bertozzi F, Bandiera T, Galietta L J V. Pharmacological potentiators of the calcium signaling cascade identified by high-throughput screening. PNAS Nexus 2:pgac288, 2022
40. Guidone D, Buccirossi M, Scudieri P, Genovese M, Sarnataro S, De Cegli R, Cresta F, Terlizzi V, Planelles G, Crambert G, Sermet I, Galietta L J. Airway surface hyperviscosity and defective mucociliary transport by IL-17/TNF-α are corrected by β-adrenergic stimulus. JCI Insight 7:e164944, 2022
41. Lazarowski E R, Boucher R C. Purinergic receptors in airway hydration. Biochem Pharmacol 187:114387, 2021
42. Ma H P, Eaton D C. Acute regulation of epithelial sodium channel by anionic phospholipids. J Am Soc Nephrol 16:3182-3187.
43. Shei R J, Peabody J E, Kaza N, Rowe S M. The epithelial sodium channel (ENaC) as a therapeutic target for cystic fibrosis. Curr Opin Pharmacol 43:152-165, 2018

1. A positive modulator of Ca²⁺ signaling according having general formula (I) or general formula (II) or general formula (III) or general formula (IV): 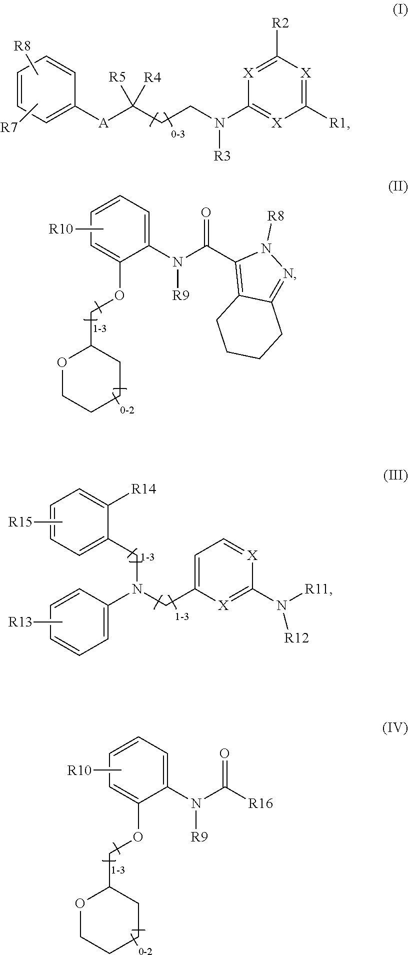
wherein in general formula (I):
X is C or N, with the proviso that at least one X is N; A is O, S, NH, N—C₁₋₆ alkyl;
R₁ is an heteroaromatic ring optionally substituted with one or more substituents selected from C₁₋₆ alkyl, C₃₋₆ cycloalkyl, halogen, OH, OC₁₋₆ alkyl;
R₂ is H, C₁₋₆ alkyl, C₃₋₆ cycloalkyl, NH₂, NHC(═O)C₁₋₆alkyl, NHC₁₋₆ alkyl, N(C₁₋₆ alkyl)₂;
R₃ is H, C₁₋₆ alkyl, C₃₋₆ cycloalkyl;
R₄ and R₅ are each independently H or C₁₋₆ alkyl; or R₅ is linked to R₃ to form a 5- or 6-membered heterocyclic ring;
R₆ and R₇ are each independently H, C₁₋₆ alkyl, OC₁₋₆ alkyl, C₃₋₆ cycloalkyl, halogen, CN;
wherein in general formula (II):
R₈ and R₉ are each independently selected from H, C₁₋₆ alkyl and C₃₋₆ cycloalkyl;
R₁₀ is H, C₁₋₆ alkyl, OC₁₋₆ alkyl, C₃₋₆ cycloalkyl, halogen, CN;
wherein in general formula (III):
X is C or N, with the proviso that at least one X is N;
R₁₁ and R₁₂ form together with the N to which are linked a pyrrolidine ring;
R₁₃ and R₁₅ are each independently selected from H, C₁₋₆ alkyl, OC₁₋₆ alkyl, C₃₋₆ cyclo-alkyl, halogen, CN;
R₁₄ is H, C₁₋₆ alkyl, (CH₂)ₙOH, (CH₂)ₙNH₂, (CH₂)ₙOC₁₋₆ alkyl, (CH₂)ₙNHC₁₋₆ alkyl, (CH₂)ₙN(C₁₋₆ alkyl)₂, wherein n is 1 to 6;
wherein in general formula (IV):
R₉ is H, C₁₋₆ alkyl or C₃₋₆ cycloalkyl;
R₁₀ is H, C₁₋₆ alkyl, OC₁₋₆ alkyl, C₃₋₆ cycloalkyl, C₁₋₆ fluoroalkyl, OC₁₋₆ fluoroalkyl, halogen, CN or an heteroaromatic ring;
R₁₆ is a mono- or bicyclic cycloalkyl, mono- or bicyclic heterocycloalkyl, mono- or bicyclic cycloalkenyl or mono- or bicyclic heterocycloalkenyl optionally substituted with one or more substituents each independently selected from C₁₋₆ alkyl, OC₁₋₆ alkyl, C₃₋₆ cycloalkyl, halogen, aromatic or heteroaromatic ring;
and any tautomeric form, enantiomer, isotopic variant, salt and solvate thereof.

2. The positive modulator of Ca²⁺ signaling of general formula IV according to claim 1, wherein R₁₆ is selected from cyclopropyl, cyclobutyl, cyclopentyl, cyclohexyl, cycloheptyl, cyclooctyl, tetrahydrofurane, tetrahydropyrane, piperidine, morpholine, spiro[2.3]hexane, bicyclo[3.1.0]hexane, 5-oxaspiro[2.4]heptane, 2-azabicyclo[2.2.1]heptane, bicyclo[2.2.1]heptene each of said ring being optionally substituted with one or more substituents each independently selected from C₁₋₆ alkyl, OC₁₋₆ alkyl, C₃₋₆ cycloalkyl, halogen, OH, CN, optionally substituted phenyl, optionally substituted thiophene, optionally substituted pyridine, optionally substituted pyrrole, optionally substituted pyrazole, optionally substituted imidazole or optionally substituted thiazole.

3. The positive modulator of Ca²¹ signaling, according to claim 1 being selected from the following compounds: 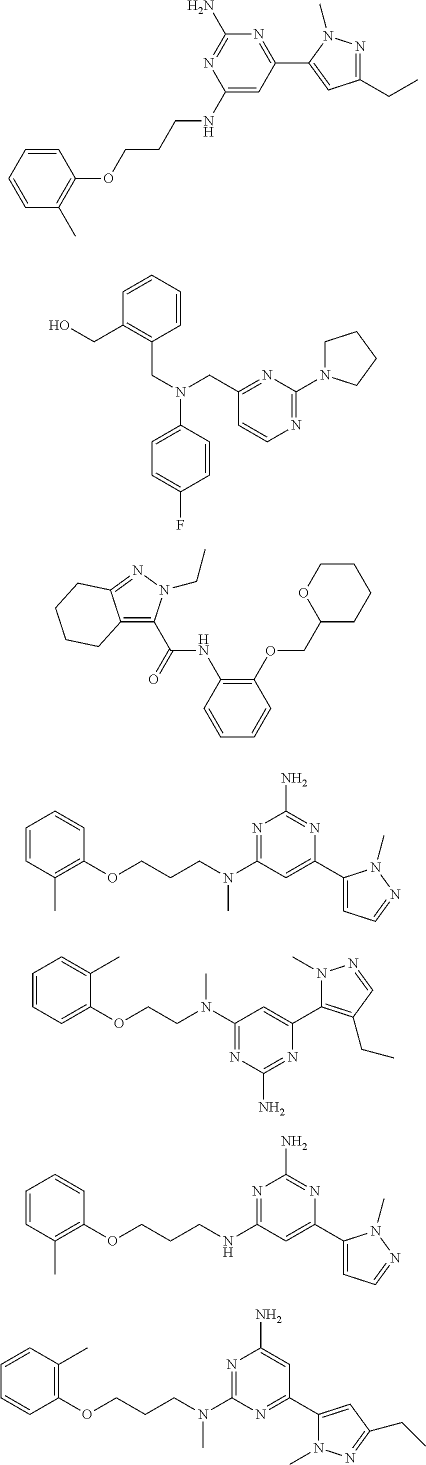 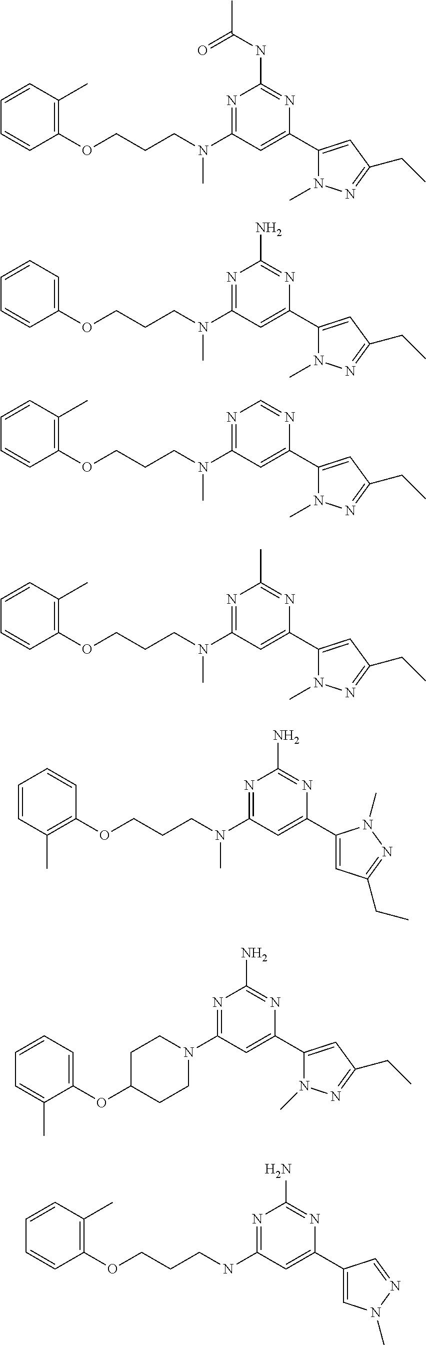
and any tautomeric form, enantiomer, isotopic variant, salt and solvate thereof.

4. The positive modulator of Ca²⁺ signaling, according to claim 2 being selected from the following compounds: 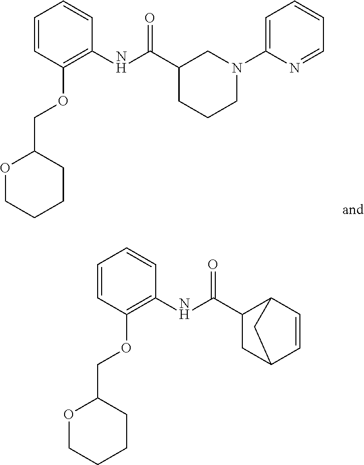 [SMILES: O=C(Nc1ccccc1OCC1CCCCO1)C1CCCN(c2ccccn2)C1 ; O=C(Nc1ccccc1OCC1CCCCO1)C1CC2C=CC1C2]
and any tautomeric form, enantiomer, isotopic variant, salt and solvate thereof.

5. The positive modulator of Ca²⁺ signaling according to claim 1 being an allosteric potentiator of the purinergic receptor P2RY2 and/or of ITPR, preferably said ITPR is ITPR1.

6. A compound of general formula (I) or of general formula (II) or of general formula (III) or of general formula (IV) as defined in claim 1 wherein the compound is a positive modulator of Ca²⁺ signaling.

7. A method for the treatment and prevention of diseases and conditions affected by modulation of Ca²⁺ signaling, comprising administering an effective amount of a positive modulator of Ca²⁺ signaling according to claim 1 to a subject in need thereof, and said diseases and conditions are optionally associated with impaired mucociliary clearance respiratory diseases and conditions, ocular diseases and conditions, spinocerebellar ataxia, dry mouth (xerostomia), intestinal hypermobility, cholestasis, and Gillespie syndrome (GLSP; OMIM #206700).

8. The method according to claim 7, wherein the respiratory diseases and conditions are selected from cystic fibrosis, chronic obstructive pulmonary disease (COPD), chronic bronchitis, emphysema, bronchiectasis, including non-cystic fibrosis bronchiectasis, asthma and primary ciliary dyskinesia, respiratory tract infections, lung carcinoma;
the spinocerebellar ataxia is spinocerebellar ataxia caused by ITPR1 defective function, spinocerebellar ataxia type 29 (SCA29) or spinocerebellar ataxia type 15 (SCA15);
the dry mouth (xerostomia) results from Sjorgen syndrome, radiotherapy treatment or xerogenic drugs;
the intestinal hypermobility is associated with gastric dyspepsia, gastroparesis, chronic constipation or irritable bowel syndrome; and
the ocular disease is dry eye disease.

9. A pharmaceutical composition comprising one or more positive modulators of Ca²⁺ signaling compounds according to claim 1 together with a pharmaceutically acceptable excipient.

10. The pharmaceutical composition according to claim 9 further comprising one or more additional pharmaceutical agents, optionally selected from mucolytic agents, bronchodilators, antibiotics, anti-infective agents, CTFR modulators and anti-inflammatory agents.

11. (canceled)

12. (canceled)
